(19) United States
(12) Patent Application Publication
(10) Pub. No.: US 2022/0105510 A1
(43) Pub. Date: Apr. 7, 2022

(54) MICROFLUIDIC CELL CULTURE DEVICES

(71) Applicant: Massachusetts Institute of Technology, Cambridge, MA (US)

(72) Inventors: Duncan A. O'Boyle, Cambridge, MA (US); Linda Griffith, Cambridge, MA (US); David Trumper, Cambridge, MA (US)

(21) Appl. No.: 17/495,594

(22) Filed: Oct. 6, 2021

Related U.S. Application Data
(60) Provisional application No. 63/088,900, filed on Oct. 7, 2020.

(51) Int. Cl. B01L 3/00

(52) U.S. Cl. CPC B01L 3/502707; B01L 2200/0689; B01L 2200/12; B01L 2200/16; B01L 2300/0887; B01L 2300/0819; B01L 2300/123; B01L 2400/049; B01L 2300/069

(57) ABSTRACT

Materials and methods of making have been developed for mass production of thermoplastic microfluidic chips. An elastomer diaphragm with a stress relieving feature can be used in microfluidic valves, pump diaphragms, and diaphragm micropumps. An optimized pump chamber design for complete fluid displacement and chamber geometry are provided. Microfluidic pressure regulators use a pneumatically actuated elastic membrane in a back-pressure regulator configuration. Microfluidic accumulators store pressurized fluid in a microfluidic chip. Removable caps for cell culture and a quick release top are described. Methods to incorporate hydrogels and ECM scaffolds have been developed. Electro pneumatic manifolds connect and control of multiple microfluidic devices vertically or on a rotary mechanism.

### CROSS REFERENCE TO RELATED APPLICATIONS

This application claims the benefit of and priority to U.S. Provisional Application No. 63/088,900 filed Oct. 7, 2020, which is hereby incorporated herein by reference in its entirety.

### FIELD OF THE INVENTION

The present invention is generally in the field of manufacturing processes and components used in microfluidic cell culture devices.

### BACKGROUND OF THE INVENTION

Microfluidics refers to the behavior, precise control, and manipulation of fluids that are geometrically constrained to a small scale (typically sub-millimeter). It is a multidisciplinary field that involves engineering, physics, chemistry, biochemistry, nanotechnology, and biotechnology. Microfluidics has practical applications in the design of systems that process low volumes of fluids to achieve multiplexing, automation, and high-throughput screening.

Microfluidic cell culture integrates knowledge from biology, biochemistry, engineering, and physics to develop devices and techniques for culturing, maintaining, analyzing, and experimenting with cell cultures at the microscale. It merges microfluidics, a set of technologies used for the manipulation of small fluid volumes (μL, nL, pL) within artificially fabricated microsystems, and cell culture, which involves the growth and proliferation of cells in a controlled laboratory environment. Microfluidics has been used for cell biology studies as the dimensions of the microfluidic channels are well suited for the physical scale of cells (in the order of magnitude of micrometers). For example, eukaryotic cells have linear dimensions between 10-100 μm which falls within the range of microfluidic dimensions. A key component of microfluidic cell culture is being able to mimic the cell microenvironment which includes soluble factors that regulate cell structure, function, behavior, and growth. Another important component for the devices is the ability to produce stable biomolecular gradients that are present in vivo as these gradients play a significant role in understanding chemotactic, durotactic, and haptotactic effects on cells. Traditional two-dimensional (2D) cell culture is cell culture that takes place on a flat surface, e.g. the bottom of a well-plate, and is known as the conventional method. While these platforms are useful for growing and proliferating cells to be used in subsequent experiments, they are not ideal environments to monitor cell responses to stimuli as cells cannot freely move or perform functions as observed in vivo that are dependent on cell-extracellular matrix material interactions. To address this issue many methods have been developed to create a three-dimensional (3D) native cell environment. Since the advent of poly(dimethylsiloxane) (PDMS) microfluidic device fabrication through soft lithography microfluidic devices have progressed and have proven to be very beneficial for mimicking a natural 3D environment for cell culture.

Recent advances in cell biology, microfabrication and microfluidics have enabled the development of microengineered models of the functional units of human organs, known as organs-on-a-chip (OOC) that could provide the basis for preclinical assays with greater predictive power. Early embodiments have been described and commercialized. For example, U.S. Pat. No. 6,197,575 to Griffith, et al., describes a micromatrix and a perfusion assembly suitable for seeding, attachment, and culture of complex hierarchical tissue or organ structures. U.S. Pat. No. 8,318,479 to Inman, et al., describes a system that facilitates perfusion at the length scale of a capillary bed suitable for culture and assaying in a multiwell plate format. U.S. Application Publication Nos. US 2016/0377599 and US 2017/0227525 A1 describe organ microphysiological systems with integrated pumping, leveling and sensing.

These platforms, termed microphysiological systems (MPSs), are designed to mimic physiological functions by integrating tissue engineering principles with microfabrication or micromachining techniques for recapitulating 3D multicellular interactions and dynamic regulation of nutrient transport and/or mechanical stimulation (Huh D, et al., Lab Chip, 12(12):2156-2164 (2012); Sung J H, et al. Lab Chip 13(7):1201-1212 (2013); Wikswo J P, et al., Exp Biol Med (Maywood) 239(9):1061-1072 (2014); Livingston CA, et al., Computational and Structural Biotechnology Journal 14:207-210 (2016); Yu J, et al., Drug Discovery Today, 19(10):1587-1594 (2014); Zhu L, et al. Lab Chip, 16(20):3898-3908 (2016)). While significant advances have been made in the development of individual MPS (e.g., cardiac, lung, liver, brain) (Roth A, et al., Adv Drug Deliver Rev, 69-70:179-189 (2014); Huebsch N, et al. Scientific Reports, 6:24726 (2016); Domansky K, et al. Lab Chip 10(1):51-58 (2010)), efforts towards the interconnection of MPS are still in their infancy, with most studies primarily focused on basic viability and toxicity demonstrations (Oleaga C, et al. Sci Rep 6:20030 (2016); Esch M B, et al., Lab Chip 14(16):3081-3092 (2014); Maschmeyer I, et al., Lab Chip 15(12):2688-2699 (2015); Materne E M, et al. J Biotechnol 205:36-46 (2015); Loskill P, et al., Plos One 10(10):e0139587 (2015)). However, lack of clinical efficacy, rather than toxicity, was identified as the leading cause of drug attrition in Phase II and III clinical trials (the most costly stage) (Kubinyi H, Nat Rev Drug Discov 2(8):665-668 (2003); Cook D, et al. Nat Rev Drug Discov 13(6):419-431 (2014); Denayer T, et al., New Horizons in Translational Medicine, 2(1):5-11 (2014)). Major contributing factors include incomplete understanding of disease mechanisms, the lack of predictive biomarkers, and interspecies differences. There is an urgent unmet need in drug development due to the need for humanized model systems for target identification/validation and biomarker discovery.

While toxicology and pharmacodynamic studies are common applications, pharmacokinetic studies have been limited in multi-MPS platforms. Moreover, current multi-MPS systems may employ a closed format associated with traditional microfluidic chips for operating with very small fluid volumes (Anna SL, Annu. Rev. Fluid Mech. 48, 285-309 (2016)). Current fabrication processes for these systems require the use of castable elastomeric polymers (Halldorsson S, et al., Biosens. Bioelectron. 63, 218-231 (2015)).

International Patent Application No. PCT/US2019/030216 “Pumps and Hardware For Organ-On-Chip Platforms” Massachusetts Institute of Technology describes a number of different improvements to fluid handling, including pumps, valves, and devices to control and actuate these systems.

Materials and New Fabrication Methods to Make these Devices

Some considerations for microfluidic devices relating to cell culture include: fabrication material (e.g., polydimethylsiloxane (PDMS), polystyrene), bulk material properties (e.g., optical clarity, surface properties), fabrication method (e.g., injection molding, hot embossing), culture region geometry, method of delivering and removing media, and flow configuration using passive methods (e.g., gravity-driven flow, capillary pumps, Laplace pressure based ‘passive pumping’) or a flow-rate controlled device (i.e., perfusion system). The flexibility of microfluidic devices greatly contributes to the development of multi-culture studies by improved control over spatial patterns. Closed channel systems made of PDMS are most commonly used because PDMS has traditionally enabled rapid prototyping of biocompatible microdevices. For example, mixed co-culture can be achieved in droplet-based microfluidics easily by a co-encapsulation system to study paracrine and juxtacrine signaling. Two types of cells are co-encapsulated in droplets by combining two streams of cell-laden agarose solutions. After gelation, the agarose microgels serve as a 3D microenvironment for cell co-culture. Segregated co-culture in microfluidic channels is used to study paracrine signaling. Human alveolar, epithelial cells and microvascular endothelial cells can be co-cultured in compartmentalized PDMS channels, separated by a thin, porous, and stretchable PDMS membrane to mimic alveolar-capillary barrier.

Fabrication material is crucial in the design of a cell culture device as not all polymers are biocompatible, with some materials such as PDMS causing undesirable adsorption or absorption of small molecules. Additionally, uncured PDMS oligomers can leach into the cell culture media, which can harm the microenvironment. As an alternative to PDMS, there have been advances in the use of thermoplastics (e.g., polystyrene, polysulfone, PMMA, COC) as a replacement material. These materials provide good optical clarity and small feature reproduction without the tradeoff of interaction with small biomolecules. The ability to fabricate devices using these materials poses some unique challenges which has inhibited their ubiquity in the microfluidics community.

Fabrication method is also critical in successfully creating a microfluidic device. PDMS devices are usually molded and plasma bonded to a glass microscope slide, a process that is not feasible for thermoplastic polymers. Lamination of optically clear thermoplastic microfluidic devices often requires expensive equipment (e.g., ultrasonic welding, laser welding) and is prone to low strength and unreliable bonds between the device and the optical window.

The control of fluids pressures and flowrates on the chip is critical for mimicking in vivo fluidic conditions. This can be done using gravity based flow, on-chip pumps, or external pumps such as syringe pumps. All existing pumping platforms either allow for the fluid pressure or fluid flowrate to be controlled. It is desirable to have control over the fluid pressure and the

Spatial organization of cells in microscale devices largely depends on the culture region geometry for cells to perform functions in vivo. For example, long, narrow channels may be desired to culture neurons. The perfusion system may also affect which geometry is selected. For example, in a system that incorporates syringe pumps, channels for perfusion inlet, perfusion outlet, waste, and cell loading would need to be added for the cell culture maintenance. Perfusion in microfluidic cell culture is important to enable long culture periods on-chip and to enable cell differentiation.

It is therefore an object of the present invention to provide new materials and methods for manufacturing thermoplastic microfluidic devices with improved optical clarity, biocompatibility, and integrated flexible membranes as an easy-to-manufacture alternative to polydimethylsiloxane (PDMS).

It is another object of the present invention to provide improvements to fluid handling in microfluidic devices using thin elastomer membranes.

It is a further object of the present invention to provide improved pump chambers and diaphragms for use in pneumatically actuated pumps for microfluidic devices, that induce lower stresses and are more accurate.

It is another object of the invention to provide optimized low-volume valve geometries that enhance fluid sealing pressures.

It is still another object of the invention to provide hydraulic accumulators for storing fluid volume under pressure, and back pressure regulators for controlling system pressures in a microfluidic channel.

It is a still further object of the present invention to provide improved methods of making and using hydrogel containing matrices in microfluidic devices, including ways of forming and containing hydrogel materials with removable structures as well as leveraging types of hydrogel scaffolds.

It is another object of the present invention to provide cell culture platforms that can control multiple microfluidic devices at the same time, for high-throughput studies.

It is a further object of the invention to provide disposable microfluidic chips with advanced control features and interconnects.

### SUMMARY OF THE INVENTION

### Materials and Methods of Manufacture for Microfluidic Devices

A method for bonding microfluidic devices made of cyclic olefin copolymers with integrated elastomeric membranes has been developed that enables a wide range of microfluidic components including pumps, valves, accumulators, pressure regulators, oxygenators, and pressure sensors, without the use of materials such as polydimethylsiloxane (“PDMS”). These devices can be integrated with electropneumatic control units for high throughput use with advanced process control. The process bonds optically clear, solvent resistant, and biocompatible polymers for cell culture applications. The bond strength and optical properties of these devices far exceeds that of other materials such as PDMA. These materials and methods are useful for fabrication of microfluidic systems with controlled flowrates and processes throughout the system, by means of pumps, valves, pressure regulators, accumulators, and on-chip sensing elements.”

Methods of manufacturing thin films for use in microfluidic devices have been developed. In one embodiment, a water assisted laser machining techniques for etching elastomeric polymer film, using capillary action of a water film to secure the cut pieces in place, has been developed. This method also provides a thermal sink and IR absorbing layer to control excess heat in the laser machining process. In another method, a porous vacuum chuck with negative features serves as a mold for thermoformed elastomer membranes.

A custom optical film has been developed to easily fabricate thermoplastic microfluidic chips with optical windows. The film consists of a removable polyethylene carrier film on a high temperature grade of COC that is bonded to a thin layer of elastomeric COC. The elastomeric COC is protected by a carrier film made of a polymer such as biaxially-oriented polyethylene terephthalate (MYLAR®). This film can be easily laminated in a roll lamination process or can be bonded using a thermal press or hot plate. The film can be mass produced in a roll extrusion process and cut to size using conventional laser fabrication techniques.

A custom bonding process has been developed to laminate a thin elastomer film to a microfluidic chip. The film is placed on a non-interactive carrier film like those used for thin film adhesives and supported by a flat substrate. The rigid component is aligned to the membrane and passed through a thermal laminator. The use of a carrier film and support structure enables a high strength bond to the chip without thermal warping of the membrane. New on-chip components featuring elastomer membrane

process or can be bonded using a thermal press or hot plate. The film can be mass produced in a roll extrusion process and cut to size using conventional laser fabrication techniques.

A custom bonding process has been developed to laminate a thin elastomer film to a microfluidic chip. The film is placed on a non-interactive carrier film like those used for thin film adhesives and supported by a flat substrate. The rigid component is aligned to the membrane and passed through a thermal laminator. The use of a carrier film and support structure enables a high strength bond to the chip without thermal warping of the membrane.

On-Chip Components Featuring Elastomeric Membrane

An elastomer diaphragm with a stress relieving feature has been developed to be used in microfluidic valves and pump diaphragms. This rolling diaphragm rolls to experience high displacement with limited elastic deformation. These include external rolling diaphragms, internal rolling diaphragms, shape changing diaphragms, and sideways rolling diaphragms. Diaphragm micropumps with optimized pump chambers that ensure reliable displacement volume and improved reliability have been developed. One pump chamber features a rolling diaphragm and one features a pump chamber with a predictable displacement stroke. The rolling diaphragm pump chamber uses a rolling diaphragm to displace fluid volume in a chamber. The diaphragm can be actuated using compressed gas and vacuum. Another pump chamber design is an optimized shape that guarantees complete fluid displacement from the pump chamber. The chamber geometry is designed around the elastic response of a flexible membrane under pressurized load such that the membrane retains a ring of contact with the pump chamber during a pump stroke. This feature eliminates the chance for small pockets of fluid to get trapped in the diaphragm and ensure reliable displacement volumes.

In a preferred embodiment, an elastomeric diaphragm with a stress relieving feature has been developed to be used in microfluidic valves and pump diaphragms. This rolling diaphragm rolls to experience high displacement with limited elastic deformation. These include external rolling diaphragms, internal rolling diaphragms, shape changing diaphragms, and sideways rolling diaphragms. Diaphragm micropumps with optimized pump chambers that ensure reliable displacement volume and improved reliability have been developed. One pump chamber features a rolling diaphragm and one features a pump chamber with a predictable displacement stroke. The rolling diaphragm pump chamber uses a rolling diaphragm to displace fluid volume in a chamber. The diaphragm can be actuated using compressed gas and vacuum. Another pump chamber design is an optimized shape that guarantees complete fluid displacement from the pump chamber. The chamber geometry is designed around the elastic response of a flexible membrane under pressurized load such that the membrane retains a ring of contact with the pump chamber during a pump stroke. This feature eliminates the chance for small pockets of fluid to get trapped in the diaphragm and thereby ensures reliable displacement volumes.

Microfluidic pressure regulators that use a pneumatically actuated elastic membrane as a sealing feature and compressed gas as a biasing element have been developed. In a preferred embodiment fluid builds up pressure against the elastic membrane until it overcomes the pressure exerted by the compressed gas on the other side and serves as a back-pressure regulator. In an alternative embodiment the regulator controls the fluid pressure downstream of the regulating element. The diaphragm is designed to have low stiffness so that it is not sensitive to strain energy in the membrane. The fluid begins to flow once the fluid pressure exceeds the sealing pressure. Fluid pressure can be regulated by adjusting the compressed gas source and the flow can be stabilized by adding compliance in the fluidic circuit.

Several different types of microfluidic accumulators can be used to store pressurized fluid in a microfluidic chip. In one embodiment, the accumulator uses a flexible membrane to store pressure using stored elastic energy in the membrane. In another embodiment, a microfluidic accumulator uses small dead-end microfluidic channels for trapping gas bubbles and storing volume under pressure. In a third embodiment the microfluidic accumulator uses a rolling diaphragm pressurized with air on one side and fluid stored in a reservoir.

Several on-chip pressure sensors have been developed. In one embodiment, the sensor uses an optical level or change in capacitance and deformable membrane, where deformation of the elastic membrane occurs with an increase in pressure. In another embodiment, a camera is used to measure the length of trapped gas bubbles in microfluidic channels which is proportional to the channel pressure.

Methods for Hydrogel Installation and Tissue Scaffolding

A variety of hydrogel forming techniques are described. In one embodiment, removable or dissolvable support structures are used to position the hydrogel at the time of formation, and/or to create channels in the hydrogel for fluid flow. In an alternative embodiment, foldable flaps are used to shape the hydrogel, then folded out of the way. In still another embodiment, channels are created through the creation of wedges or channels in the containers that match features on the manifolds into which they are inserted. In yet another embodiment, a slot shaped hanging drop hydrogel held in place by surface tension is used to separate media channel and change flow configurations as a function of swelling. The use of non-adhering polymers including polytetrafluoroethyelene (“PTFE”) allows for these structures to be removed without damaging the hydrogel after polymerization.

Scaffolds of various extracellular matrix (“ECM”) materials can be laser cut for use in microfluidic chips and transwell inserts. Laser cut holes can vary in size and shape from a few microns in size up to millimeters. The use of optically clear thin films allows for these scaffolds to be imageable and the hydrophobic nature allows for an ECM to be incorporated in a liquid phase.

Platforms for High Throughput Cell Culture Studies

Removable caps have been designed for use in microfluidic devices for cell culture applications. These may include optically clear windows, elastomeric features for better compliance, or an adhesive pattern on a film for improved sealing. Reservoirs for the microfluidic chip can also be designed to accommodate two-position cell culture caps and other existing cap designs. In another embodiment, a quick release top for a microfluidic chip was developed which uses a gasket compressed using a spring-loaded lever, a toggle clamp or an overcenter latch.

Electro pneumatic manifolds for stacking microfluidics devices have been developed which incorporate the devices vertically or on a rotary mechanism. These manifolds distribute pneumatic signals to multiple chips for high throughput experiments. The individual manifolds also feature a latching system to enable quick connection of the microfluidic devices to the pneumatic lines.

### BRIEF DESCRIPTION OF THE DRAWINGS

FIG. 1 shows an elastomeric film 2, approximately 25-60 microns, formed of a COC polymer such as E-140, an optical film 3, approximately 100-200 microns in thickness, formed an optically clear polymer such as COC, preferably 6013F04, with removable carrier films 1, 4, formed of a polymer such as polyethylene terephthalate (“PET”), approximately 25-60 microns thick. Labels: (1) removable carrier film (PET, ˜25-60 μM); (2) Elastomeric COC (E-140, ˜25-60 μm); (3) Optical COC (6013F04, ˜100-200 μm) and (4) removable carrier film (coated PET, ˜25-60 μm).

FIG. 2 is a view of a process of aligning elastomer COC films to a flat substrate such as a silicon wafer (11) by sending the elastomer COC membrane film (E-140, 25-60 μM) (7), preferably in combination with a protective cover film formed of a material such as a polyethylene film with a silicone release coating (˜25 μM) (8), on the flat substrate through a heated thermal roll laminator (13) heated to a temperature of about 130° C., to produce the aligned films on the microfluidic chip. The final product will typically have on top a protective film that can be removed easily, the elastomeric COC and/or polymethacrylate (PMMA) layer, microfluidic chip (9), all on a silicon wafer.

FIG. 3 is a diagram of a water assisted laser machining techniques for etching elastomeric polymer film, using capillary action of a water film. The supporting material can be IR absorbing or transmissive depending on the application. Labels: (260) Thin film elastomer; (262) water; (264) Pane of glass, germanium, sapphire, ice or IR polymer; (266) Cut parts held down with capillary force; (268) CO₂ laser.

FIGS. 4A-4D are cross-sectional views of a porous vacuum chuck with negative features that serve as a mold for thermoformed elastomer membranes (FIG. 4A), showing that vacuum deforms the elastomeric membrane into the mold (FIG. 4B), to yield a standalone thermoformed membrane (FIG. 4C), or can bonded to the manifold while hot (FIG. 4D). Labels: (270) Vacuum chuck; (272) Mold feature; (274) Elastomeric membrane; (276) Porous carbon; (278) Vacuum; (280) Add heat to reach elastomer melting point; (282) Vacuum deforms into mold; (284) Bonded membrane; (286) yields thermoformed membrane; (288) could bond to manifold while hot.

FIGS. 5A and 5B are prospective views of a rolling diaphragm showing the hoop strain. The rolling diaphragm (10) has a rolling lip (12) with a lip (14), with a hoop (16).

FIGS. 6A-6D are schematics showing different types of rolling diaphragms. FIG. 6A is an external rolling diaphragm; FIG. 6B is an internal rolling diaphragm; FIG. 6C is a shape changing diaphragm; FIG. 6D is a sideways rolling diaphragm.

FIGS. 7A-7E are schematics of the mechanism of pumping using a rolling elastomer diaphragm (20). In FIG. 7A, a pneumatic pressure source (+P) (30) is used to displace the diaphragm. In FIG. 7B, vacuum (−P) (32) is used to draw the diaphragm and fill a reservoir (34). In FIG. 7C, pressure is then applied for a displacement stroke. Before fluid aspiration, FIG. 7A; Vacuum is used to fill reservoir, FIG. 7B; chamber full of liquid, FIG. 7C; pressure is applied to chamber, FIG. 7D; end of displacement stroke FIG. 7E.

FIGS. 8A-8F are schematics of pump chambers 40, comparing an ideal pump chamber 44 with an unoptimized chamber 46. FIGS. 8A, 8B, 8C show the ideal pump chamber 44, where the diaphragm 20 maintains constant contact with the pump chamber 44 during actuation, as compared to the unoptimized chamber 46 of FIGS. 8D, 8E, and 8F, which risks trapping fluid 48 inside of the diaphragm membrane 20 causing unpredictable displacement volumes. FIG. 8G is an expanded view of the contact between the diaphragm and the pump chamber wall

FIGS. 9A-9C are schematics of a microfluidic pressure regulator 50 that uses a pneumatically actuated elastic membrane as a sealing feature and compressed gas as a bias. Fluid builds up pressure against the elastic membrane until it overcomes the pressure exerted by the compressed gas on the other side.

FIGS. 9A, 9B. The fluid begins to flow once the fluid pressure exceeds the sealing pressure. FIG. 9C. Fluid pressure can be regulated by adjusting the compressed gas source and the flow can be stabilized by adding compliance in the fluidic circuit. Labels: (60) Pressure regulator; (62) rolling diaphragm; (64) air pressure source; (66) pressure setpoint; (68) fluid; (70) side flow; (72) diaphragm chamber; (74) side sealing); (76) fluid pressure PH.

FIG. 10 is a schematic of a valve with a bonded elastic membrane and a defined sealing contact. Fluid flow can be bi-directional. Sealing lip can be a small flat surface or a rounded shape as shown. Labels: (90) valve; (92) bonded elastic membrane; (94) sealing contact; (96) sealing surface; (98) valve inlet; (100) fluid inlet; (102 and 104) valves of fluidic manifold.

FIG. 11 is a valve that has a rounded sealing feature that amplifies the sealing pressure at the inlet of the valve, showing the valve in cross section with the membrane experiencing a higher strain and contact pressure at the sealing interface. Specifications of exemplary valve—D: 1.5 mm membrane with 0.2 mm seat radius; equivalent elastic strain; Type: equivalent elastic strain; Unit: m/m; Time:1.

FIGS. 12A-12C are a teardrop shaped valve with rounded sealing surface. FIG. 12A is a perspective view of the teardrop shaped valve with a rounded sealing surface and a teardrop shape that reduces the overall volume of the valve. The teardrop shape reduces the dead volume of the valve when compared to a circular profile valve of the same size inlet. Here is a screenshot of the teardrop valve in CAD. Sealing shape in red dashed line. FIG. 12B shows the valve integrated in a pump. FIG. 12C is a cross-sectional view of the valve integrated in the pump. FIG. 12D is a graph comparing the performance of various valves (doormat, ring, teardrop, valve in FIG. 8), demonstrating that the teardrop valve exhibits improved performance over out previously designed doormat valves.

FIGS. 13A-13C are schematics of several different types of microfluidic accumulators. FIG. 13A is a schematic of an accumulator using a flexible membrane to store pressure using stored elastic energy in the membrane. FIG. 13B is schematic of a microfluidic accumulator using small dead-end microfluidic channels for trapping gas bubbles and storing volume under pressure. FIG. 13C is a schematic of a microfluidic accumulator that uses a piston pressurized with air on one side and fluid stored in a reservoir. Labels: (110) accumulator; (112) rolling diaphragm/flexible membrane; (114) air pressure; (116) fluid; (118) reservoir; (120) microfluidic accumulator; (122) small microfluidic channels; (124) gas bubbles; (132) low friction piston; (134) air pressure; (136) pressurized fluid; (138) piston bone

FIGS. 14A-14C is a microfluidic accumulator, with a diaphragm pressured with air on one side and fluid is stored in a reservoir, no volume (FIG. 14A), accumulating volume (FIG. 14B), and at capacity (FIG. 14C). Labels: (140) microfluidic accumulator; (142) rolling diaphragm; (144) pressurized air; (146) fluid; (148) reservoir.

FIGS. 15A-15B are schematics of a pressure sensor with an optical level and deformable membrane, before (FIG. 15A) or after deformation of the elastic membrane by an increase in pressure (FIG. 15B). FIGS. 15A-15C are schematics of measurement of gas bubble length trapped in microfluidic channels as detected by a camera (FIG. 15A), and images of low and higher pressure levels (FIG. 15B) where longer channels for trapping gas are more sensitive (FIG. 15C). Higher pressure levels result in shorter bubble length. Labels: (210) pressure sensor; (214) elastic membrane; (216) laser; (218) output angle; (220) membrane deflection; (222) laser output; (224) photodetector; (226) reflexive coating or material; (228) pressurized fluid; (230) microfluidic accumulator; (232) gas bubble; (234) pressure; (236) liquid; (238) camera; (240) camera image.

FIGS. 16A-16E are schematics of liquid sensing methodologies for microfluidic reservoirs where a deformable membrane is incorporated into the media reservoir under hydrostatic pressure, for changes in capacitance, resistance between contacting materials or optical properties (FIG. 16A). FIG. 16B shows the membrane deflecting under pressure. FIG. 16C shows the fluid reservoir having a clear window or side, where changes in fluid levels are measured and recorded by a camera. FIG. 16D shows a similar fluid reservoir where the camera is positioned above the reservoir. FIG. 16E is a schematic of the camera taking images of the fluids containing dye to provide for optical measurement. Labels: (241) elastic membrane; (242) fluid reservoir; (244) hydrostatic pressure; (246) sensing element (resistive, capacitive, optical, etc.) or camera; (248) clear material; (250) fluid reservoir; (252) any fluid; (254) fluid with dye or color; (256) wide FOV could sense multiple reservoirs.

FIGS. 17A-17D are schematics of removable caps for cell culture applications. An optically clear snap on cap is shown in FIG. 17A. An elastomeric feature on or under the caps adds compliance, as shown in FIG. 17B. A cap formed of an optical film with a patterned adhesive for sealing is shown in FIG. 17C. A press fit seal or compressed elastomeric feature on the underside of the cap is shown in FIG. 17D. Labels: (150) cap; (152) top of cap; (154) seal at exposed elastomer surface; (156) elastomeric surface adds compliance to help with sealing; (158) sealing lip; (160) compressed elastomeric feature; (162) microfluidic chip; (164) imaging window; (166) easy access cap; (168) held in place.

FIGS. 18A-18D are schematics of a microfluidic compartment for forming a hydrogel using support structures that are removable or dissolvable. Removable support structures are shown in FIGS. 18A, 18B; with the resulting cavities forming fluid channels in the hydrogel after removal shown in cross-section in FIG. 18C; and flow through the channels in the hydrogel in the microfluidic container in 18D. Labels: (290) hydrogel compartment; (292) hydrophobic removable support structures (i.e., PFA, PTFE); (294) container; (296) hydrogel/hydrogel chamber; (298) media; (300) pin cavity or media flow connections; (302) section; (304) swell; (306) pull pins. 294 can be inserted into a larger manifold or tube connections can be used (308).

FIGS. 19A-19D shows how fluid conveying channels can be created along the sides of a hydrogel cell culture container (FIG. 19A), filled with media (FIG. 19B), then inserted into a microfluidic device (FIG. 19C), showing how a wedge in the upper wall of both ends of the device can be fitted into the microfluidic device to create a channel (FIG. 19D). Labels: (310) hydrogel compartment; (312) wide flat channels/notch as datum+sealing features; (314) sides of compartment; (316) capsule slots into device; (318) optical film base; (319) media flow; (320) thin removable film (like sticker).

FIGS. 20A-20B are cross-sectional schematics of gels positioned next to ridged support structures that constrain the gel which swells upward to deform a compliant membrane (FIG. 20B). FIG. 20C shows a device with dissolvable posts or support structures retaining the hydrogel, which is inserted into the device through a port above the posts so that the hydrogel conforms to the shape designated by the support structures. FIG. 20D shows a cross-sectional view of the gel with the posts or support structures intact and after they have dissolved.

FIG. 20E shows the same structures as the gel swells and is constrained by the posts or support structures, until they dissolve or are removed. FIG. 20F shows the gel with posts, where the gel is over-constrained, FIG. 20G shows the gel without posts, where the gel is free to expand. Labels: (322) Phase guide; (324) sharp ridges or walls; (325) gel install port; (326) channels; (328) hydrogel; (329) dissolvable posts; (330) hyper-elastic material backing.

FIGS. 21A-21C are cross-sectional schematics of a fillable compartment with an integrated imaging window that uses a rotating flap (FIG. 21B) instead of support posts to contain the hydrogel until it solidifies (FIG. 21A), then is rotated open to allow the hydrogel to expand (FIG. 21C). Labels: (325) gel install port; (328) hydrogel; (332) optical window; (334) flaps open; (336) gel expansion; (338) axle; (340) rotating flap; (342) rotating axis.

FIGS. 22A-22D are cross-sectional schematics showing how a plug is removed following formation of a hydrogel in a compartment for culturing cells in a microfluidic device (FIG. 22A), the compartment is then connected at the top and bottom to channel nutrients and gases through the hydrogel (FIG. 22B), showing the flowing media adjacent to and through the hydrogel (FIG. 22C, 22D). Labels: (350) fillable container; (352) hydrogel; (354) swollen gel remains in capsule; (358) flow in+out; (360) media flow; (362) image from here.

FIGS. 23A-23E are cross-sectional schematics of a slot shaped hanging drop hydrogel held in place by surface tension (FIGS. 23A, 23B), the top and side views (FIGS. 23C, 23D), where the gel is swollen to separate a media channel into two channels (FIG. 23E), and the resulting flow configurations: across the top and under the drop (FIG. 23F), along the length of the drop (FIG. 23G), and along the sides and within the microfluidic device (FIG. 23H). Labels: (360) insect gel; (364) hydrogel drop; (366) long hanging drop; (368) expansion of hydrogel drop (370a and 370b) media channel; (372) top of hanging drop; (374) bottom of hanging drop; (374) obstructed flow; (376) device; (378) sides of drop (assumes sealing).

FIGS. 24A-24D are schematics of electropneumatic manifolds for stacking microfluidics devices (FIG. 24A) vertically (FIG. 24B) or on a rotary mechanism (FIGS. 24C, 24D). Labels: (190 and 200) electro pneumatic manifolds); (192) microfluidic device; (194) pressure; (196) vacuum; ((198) control unit; (202) carousel; (204) pneumatic connector; (206) micropump; (208) valves, solenoids, etc.; (210) USB control unit; (212) chip rotates out for sampling; (214) pipette access; (216) media sampling; (218) confocal imaging; (220) wide field imaging; (222) rotating control unit; (224) quick chip connect; (226) bonded microfluidic chip.

FIGS. 25A-25F are perspective views of the microchips inserted into the manifold (FIG. 25A), latched to secure in place (FIG. 25B), with clamp or lever pressed down to secure chip and compress the O-ring to ensure pneumatic connect to chip (FIGS. 25C-25F). FIGS. 25C-25F are perspective views a quick release latch for a microfluidic chip, using a compressed gasket compressed using a spring loaded lever, a toggle clamp, or an over-center latch. FIGS. 25D-25E are cross-sectional views of quick release toggle clamp (FIG. 25D) or an over-center latch (FIG. 25E). Labels: (328) Pneumatic connection to control unit; (330) top; (332) latch; (334) manifold; (336) internal pneumatic lines; (338) chips; (340) overhanging chips enables imaging; (342) open latch; (344) closed latch; (346) chip; (348) axis of lobe; (350) clamp; (170) micropump on chip; (172) rubber gasket; (174) quick release lever; (176) clamp force; (178) torsion spring; (180) one side fixed; (182) microfluidic chip; (184) gasket (loose); (186) axis of rotation; (188) electro pneumatic manifold; (190) lobe over center; (192) gasket (clamped); (194) off axis lobe.

FIGS. 26A-26D are perspective view of a standard chip format (FIG. 26A). FIG. 26A depicts the microfluidic chip with membrane bonded within it, chambered corners and reduced aspect ratio compared to microscope slides, to enhance bonding. FIG. 26B shows the vent, allowing gas to escape when the membrane is bonded to the chip. FIG. 26C is a side view showing the vents in a five layer microchip. FIGS. 26D and 26E show the chips have a raised edge that protects the optical film on the top and bottom.

### DETAILED DESCRIPTION OF THE INVENTION

### I. Definitions

The term “microfluidic” refers to a system that involves the control and manipulation of small fluid volumes in channels with dimensions on the order of a few micrometers up to a few millimeters and total system volumes on the scale of nanoliters to a few milliliters. As used herein, the term “channel” refers to a closed volume where fluid passage occurs. A channel may vary in cross sectional area and length. A channel may have square, circular or other cross-sectional shape.

The term “chip” refers to the component where microfluidic fluid manipulation occurs. A chip may be made of a wide variety of materials and can be different sizes. A “device” refers to a chip or microfluidic system that performs a function or series of functions. A device may consist of one or more chips.

As used herein, the term “hydrogel” refers to a substance formed when an organic polymer (natural or synthetic) is cross-linked via covalent, ionic, or hydrogen bonds to create a three-dimensional open-lattice structure which entraps water molecules to form a gel. Biocompatible hydrogel refers to a polymer forms a gel which is not toxic to living cells and allows sufficient diffusion of oxygen and nutrients to the encapsulated cells to maintain viability.

As used herein, the term “extracellular matrix”, “ECM” refers to the components and/or the network of extracellular macromolecules, such as proteins, enzymes, and glycoproteins, that provide structural and biochemical support of surrounding cells. The extracellular matrix includes the interstitial matrix and the basement membrane components of the ECM include proteoglycans heparan sulfate, chondroitin sulfate, keratan sulfate; non-proteoglycan polysaccharide hyaluronic acid, and proteins collagen, elastin, fibronectin, and laminin.

As used herein, the term “extracellular matrix-binding peptide” refers to a synthetic peptide with affinity to ECM components.

As used herein, the term “hydrogel matrix” typically refers to the network of cross-linked polymers forming the hydrogel. The hydrogel matrix may or may not include the binders.

The term “scaffold” in the relevant sections is an insert or component which provides support for tissue constructs and ECM components.

The term “media” refers to a fluid that is used for cell culture and contains nutrients, growth factors, or other biomolecules that are included to grow and proliferate cells.

As used herein, the term “biodegradable”, in the context of polymer, refers to a polymer that will degrade or erode by enzymatic action and/or hydrolysis under physiologic conditions to smaller units or chemical species that are capable of being metabolized and/or eliminated.

As used herein, the term “fluid” refers to a material that is able to flow and is not solid. For example, air and water would both be considered fluids.

As used herein, the term “permeable” refers to the ability for a specific chemical species to transport through a material. For example, a material may be oxygen permeable or water permeable.

The term “pneumatic” refers to a system which uses air or vacuum pressure for operation. As used herein, the term “electropneumatic” refers to a pneumatic system that relies on electrically actuated valves and pressure regulators to control pressure and vacuum signals.

An actuator is a component of a device that is responsible for moving and controlling a mechanism or system, for example by opening a valve. In simple terms, it is a “mover”. An actuator requires a control signal and a source of energy to perform a mechanical action.

The term “interconnect” refers to the point of connection between two devices where electrical signals or fluids can transfer from one device to another. The interconnect can be coupled and decoupled using some sort of mechanism.

The term “gasket” refers to a compressible material that when compressed between two other components makes a reliable and fluid-tight seal.

The term “compliant” or “compliance” refers to a material or system's ability to respond to a force or loading condition. A compliant system is flexible and allows for the translation of forces in the system. Compliance is the inverse of stiffness in a mechanical system.

The term “over center” refers to a stable physical state and position of a mechanism. More force is required to reverse the position of the mechanism than is required to keep it in the over center state.

As used herein, the term “film” refers to a thin polymer material that is usually produced on a roll. A “film” is generally 25-500 microns in thickness and can vary in material properties. A “co-extruded film” is a film that consists of multiple materials that are made of different materials. A “carrier film” is a film that serves as a supporting or protective material for another film.

The term “manifold” refers to an interconnection device for pneumatic or fluid connections. A manifold consists of internal channels that distribute pressure or vacuum to another device. A manifold may or may not include integrated valves and actuators. A manifold typically refers to a component that directs and distributes air and vacuum, but other fluids may be used. A manifold may be made of a variety of materials including polymers and metals. A manifold may be made using a range of fabrication methods including assembly with fasteners, bonding, and 3D printing.

The term “high throughput” refers to the ability of a system to control more than one device or component at a time. For cell culture a high throughput system will preferably allow for tens to hundreds of devices to be controlled simultaneously.

As used herein, the term “regulator” or “pressure regulator” refers to a component that stabilizes and controls a pressure to a setpoint value. The term “regulate” describes the functional output of a regulator. A “backpressure regulator” controls the pressure prior to the regulation element. A “forward pressure regulator” controls the pressure after the regulating element. A “differential pressure regulator” controls the pressure difference across the regulating element.

As used herein, the term “accumulator” refers to a component that stores a volume of fluid under pressure. An accumulator allows for fluid volume to be temporarily stored in a system and serves as a stabilizing element for dynamic changes in pressure and flowrate. An accumulator may store fluid volume under uniform pressure, or the pressure may change based on how much volume is in the accumulator. An accumulator may be a passive or actively controlled component.

A “valve” is a component that creates a seal between a fluid and solid interface. A valve prevents or limits the flow of fluid. A “doormat valve” is a valve that uses a thin flap over a flat surface to seal over one or more fluidic inlets or outlets centered in the flat surface.

As used herein, the term “sensor” refers to a component that is used measure a physical property of a system. A sensor may directly measure the property or infer the measurement from some other observed phenomena.

As used herein, the term “dead volume” refers to any volume in a chip or device that is deemed unnecessary or not useful.

A “reservoir” is a component that stores fluid volume.

A “cap” is a component that is used to cover and seal a component. A cap may be used to cover a reservoir but may be used to cover other components as well.

As used herein, the term “tissue compartment” refers to the region of a device where cells are cultured. The tissue compartment may consist of a hydrogel or other ECM material and may vary in size and shape. Different tissues may be used.

As used herein, the term “to deflect” refers to a movement by a planar object, such as an elastomeric membrane, in which a portion of the object moves away from, i.e., deflects, from the plane encompassing the surface area of the object.

As used herein, the term “membrane” refers to a thin film of material that may be permeable, semi-permeable, or impermeable depending on application. A membrane may be made of a variety of materials including COC, polycarbonate, and PTFE for example. A membrane may be stiff or flexible depending on application.

The term “bond” or “bonded” refers to the state of two materials that are joined due to covalent molecular bonds, crosslinking of polymers, or some other molecular adhesion force. A bond may be generated with solvents, surface activation using plasma, heat, pressure, and time.

The term “machining” refers to any subtractive fabrication process by which material is removed from a substrate.

The term “fixture” refers to a component that holds another component or device in place for some other operation.

The term “chuck” refers to a fixture that holds onto a flat surface.

The term “optically clear” and “optical clarity” refers to the transparency of materials over a wide range of wavelengths. An optically clear material will have about 95% transmission from the ultraviolet to the near infrared spectrum and will have a refractive index similar to glass.

As used herein, the term “displacement volume” or “displacement stroke” refers to an actuation parameter describing a volume of fluid displaced per one action (stroke) of the pump. It may be fragmented to describe the volume displaced per action of each one of the valves or pump chambers in a valve-pump chamber-valve configuration pump, or by the action of the entire pump. The displacement volume may also be fragmented to describe the volume displaced by the fluidic side, pneumatic side, or on both sides, of the valve per one valve action (stroke).

As used herein, the term “sealing pressure” refers to pressure which is at least the difference between pressure at contact and pressure required to make contact (sealing pressure=(pressure at contact)−(pressure required to make contact)).

As used herein, the term “body” in the context of an actuator refers to an object of a three-dimensional shape with an axis of symmetry, such as symmetry about a horizontal axis, a vertical axis, both, or at an angle. The body typically includes at least one set of two protruding portions in opposition to one another and symmetrical to one another along the vertical axis of symmetry. The body may include more than one set of the two portions, such as two sets, three sets, four sets, etc. The two protruding portions may be three-dimensional objects in the shape of letters I, L, P, etc. For example, the body may be I-shaped, which includes one set of two protruding portions, where each end of the I-shaped body contacts a plane parallel to the vertical axis of summery. In another example the body may be U-shaped, which includes one set of two protruding portions in the shape of the letter L, where each of the protrusions is positioned opposite to the other. Typically, the ends of the protrusions in this example contact the same plane perpendicular to the vertical axis of symmetry. The body may have a cross-sectional area in the shape of pyramid, an oblong, a square, a rectangle, a circle, or any other shape.

A thermoplastic is a polymer material that melts at a specific temperature and is able to flow in the melted state. At a certain temperature a thermoplastic will reach a “glass transition” where the molecular bonds are mobile and the material is in motion at the molecular scale. A thermoplastic can repeat these transitions multiple times.

An elastomer is a polymer that is very elastic, lightly cross-linked and either amorphous or semi-crystalline with a glass transition temperature well below room temperature. They can be envisaged as one very large molecule of macroscopic size. The crosslinks completely suppress irreversible flow but the chains are very flexible at temperatures above the glass transition, and a small force leads to a large deformation (low Young's modulus and very high elongation at break when compared with other polymers). Elastomers can be classified into three broad groups: diene, non-diene, and thermoplastic elastomers. Diene elastomers are polymerized from monomers containing two sequential double bonds. Typical examples are polyisoprene, polybutadiene, and polychloroprene. Nondiene elastomers include, butyl rubber (polyisobutylene), polysiloxanes (silicone rubber), polyurethane (spandex), and fluoro-elastomers.

Non-diene elastomers have no double bonds in the structure, and thus, crosslinking requires other methods than vulcanization such as addition of trifunctional monomers (condensation polymers), or addition of divinyl monomers (free radical polymerization), or copolymerization with small amounts of diene monomers like butadiene. Thermoplastic elastomers such as SIS and SBS block copolymers and certain urethanes are thermoplastic and contain rigid (hard) and soft (rubbery) repeat units. When cooled from the melt state to a temperature below the glass transition temperature, the hard blocks phase separate to form rigid domains that act as physical crosslinks for the elastomeric blocks. Manufacturing elastomeric parts is achieved in one of four ways: extrusion, injection molding, transfer molding, or compression molding.

A hydrogel is a cross-linked polymeric network that swells and retains a significant fraction of water within its structure, but will not dissolve in water. Most hydrogels are natural materials such as the extracellular matrix extract MATRIGEL® or synthetic hydrogels such as those described in PCT/US2020/044067 “Synthetic Hydrogels for Organogenesis” by Massachusetts Institute of Technology. The ability of hydrogels to absorb water arises from hydrophilic functional groups attached to the polymeric backbone, while their resistance to dissolution arises from cross-links between network chains.

PHASEGUIDES® are commercially available meniscus pinning barriers. They enable precise, barrier-free definition of culture matrices and cells in 3D, supporting cell-cell interactions and unprecedented imaging and quantification.

Use of the term “about” is intended to describe values either above or below the stated value in a range of approx. +/−10%; in other embodiments the values may range in value either above or below the stated value in a range of approx. +/−5%

### II. New Materials and Methods of Manufacturing Thermoplastic Microfluidic Devices

A. Cyclic Olefin Copolymer (“COC”) Elastomer Bonding Process

The material used in most microfluidic systems, PDMS, polydimethylsiloxane, also known as dimethylpolysiloxane or dimethicone, belongs to a group of polymeric organosilicon compounds that are commonly referred to as silicones. PDMS is the most widely used silicon-based organic polymer due to its versatility and properties leading to a manifold of applications. It is transparent at optical frequencies (240 nM-1100 nM), which facilitates the observation of contents in micro-channels visually or through a microscope. It has a low autofluorescence and it is considered as biocompatible (with some restrictions). PDMS bonds tightly to glass or another PDMS layer with a simple plasma treatment. This allows the production of multilayer PDMS devices to take advantage of the technological possibilities offered by glass substrates, such as the use of metal deposition, oxide deposition or surface functionalization. PDMS is deformable, which allows the integration of microfluidic valves using the deformation of PDMS micro-channels, the easy connection of leak-proof fluidic connections and its use to detect very low forces like biomechanics interactions from cells. PDMS is inexpensive compared to previously used materials (e.g. silicon). PDMS is also easy to mold, because, even when mixed with cross-linking agent, and remains liquid at room temperature for many hours. PDMS is gas permeable. It enables cell culture by controlling the amount of gas through PDMS or dead-end channels filling (residual air bubbles under liquid pressure may escape through PDMS to balance atmospheric pressure).

However, PDMS issues for microfluidic applications include absorption of hydrophobic molecules, and difficulties in performing metal and dielectric deposition on PDMS. This severely limits the integration of electrodes and resistors. Moreover, PDMS ages, therefore after a few years the mechanical properties of this material can change. For drug screening, problems arise from PDMS since PDMS adsorbs hydrophobic molecules and can release some molecules from a bad cross-linking into the liquid. PDMS also is permeable to water vapor which makes evaporation in PDMS device hard to control. PDMS is sensitive to the exposure to some chemicals. These problems make PDMS unsuitable for drug screening and development.

Elastomeric materials such as those available from TOPAS® Advanced Polymers GmbH Raunheim Germany can be used to make elastomeric membranes that do not have the same problems as PDMS membranes. These materials are described in WO2011129869, “Melt blends of amorphous cycloolefin polymers and partially crystalline cycloolefin elastomers with improved toughness”. The TOPAS® COC resins are a chemical relative of polyethylene and other polyolefin plastics, are ultra-pure, crystal-clear and UV transparent, glass like materials, with broad global regulatory compliance. They are amorphous, with heat resistance in packaging film, sterilizable, thermoformable and shrink benefits. They have barrier properties to moisture, alcohols and acids.

Numerous advantages and uses are described herein in barrier, optical window, pumping and sensor applications.

A method of bonding COC materials (primarily TOPAS® 8007s04 or TOPAS® 6013f04 with TOPAS® E-140) together using a thin film of elastomeric material and a well-controlled thermal process involves clamping flat substrates together using a simple self-leveling clamp and then bonding inside of an oven. The bonding process occurs at 84° C., the melting point of the elastomeric layer and preferably above the glass transition temperature of the rigid substrates. This overlap in glass transition temperatures guarantees a strong bond. The heating process involves heating the parts up to 84° C. slowly in the oven and then rapidly cooling them at 4° C. Although the heating process reaches the melting point of the elastomer, no material flows out of the bonded regions and unsupported elastomeric features are still retained. Further, little to no channel deformation is observed. The bond can also be done with COC elastomer to glass and COC elastomer to PMMA. Plasma activation improves bond strength for all material combinations.

These materials can also be produced as an easy to bond optical film made of a hybrid of the TOPAS 6013f-04 and E-140 grades of COC. In a preferable configuration the film can be mass produced as an 8 mil (1 mil=0.001″) thick layer of o 6013f-04 bonded to 2 mil of the E-140 resin. The 6013 side is protected with a polyethylene carrier film that is 2 mil thick and the E-140 side is on a high temperature Mylar film that is also 2 mil thick. These 4 layers provide a sterile film that can be cut to size for bonding on top of microfluidic chips. The mylar film is easily removed prior to bonding and the Polyethylene protective film can be removed prior to imaging. The material can be mass produced as a roll of material for fabrication of many microfluidic chips in a production environment.

Thermal bonding of thin elastomer films and a co-extruded 6013/E-140 film using a heated laminator is also possible. The process involves aligning thin film to the chip so that the E-140 is in contact with the bonded plane and passing the chip through a laminator. The E-140 is held on a PET carrier film with a silicone release liner and supported on a flat thin substrate, typically, a silicon wafer. The wafer provides support so that the membrane or thin film does not warp during the bonding process.

In one embodiment, the laminated films consists of four polymer films designed for application in bonding microfluidics. These are as follows:

1. 2 mil thick layer of high temperature Mylar (PET) to protect E-140 prior to bonding. Prevents dust, scratching, and contamination prior to bonding. Removable by hand.
2. 2 mil layer of TOPAS E-140 bonded to 6013F-04 Layer. Used as an easy to melt and bond layer.
3. 8 mil layer of TOPAS 6013F-04 used as an optical material. The thickness of the layer can be altered in case more stiffness or reduced thickness is desired. 8 mil is a good balance between imaging abilities and film strength.
4. 2 mil PE film. The PE film is easily removed and serves to protect the optical material from scratches.

Note that 1 mil=0.001″ and is the thickness measurement standard for thin optical films.

This material provides a significant improvement to the ability to bond COC microfluidic chips and allows for commercial lamination processes to bond devices at scale. The bond strength of this film to COC is around 28 psi channel pressure. The film also bonds to glass and PMMA like polymers.

The bonding process retains the optical clarity (from 280-800 nm) of the COC materials while providing a high bond. This process is also a safer and less equipment intensive solution to bonding parts in the lab. Other methods of bonding COC usually involve heated presses or cyclohexane, a highly flammable and toxic organic solvent.

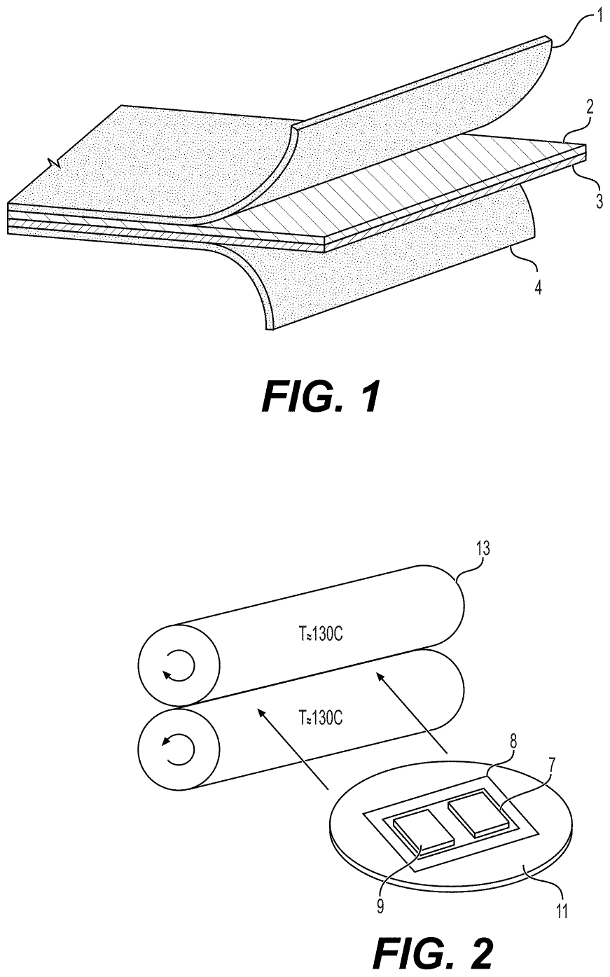

FIG. 1 shows an elastomeric film 2, approximately 25-60 microns, formed of a COC polymer such as E-140, an optical film 3, approximately 100-200 microns in thickness, formed an optically clear polymer such as COC, preferably 6013F04, with removable carrier films 1, 4, formed of a polymer such as polyethylene terephthalate (“PET”), approximately 25-60 microns thick.

FIG. 2 is a view of a process of aligning elastomer COC films 7 to a flat substrate such as a silicon wafer 11 by sending the film 7, preferably in combination with a protective cover film 8 formed of a material such as a polyethylene film with a silicone release coating, on the flat substrate 11 through a heated roll laminator 13 heated to a temperature of about 130° C., to produce the aligned films on the microfluidic chip 9. The final product will typically have on top a protective film that can be removed easily, the elastomeric COC and/or polymethacrylate (PMMA) layer, microfluidic chip, all on a silicon wafer.

B. Water Assisted CO₂ Laser Machining of Thin Elastomer Films

A process to laser machine thin elastomer films and other polymer films with minimal heat damage has been developed. The laser method involves laminating a layer of polymer onto a thin film of water using capillary action. The water layer serves to absorb stray heat and IR and acts as a workholding feature for the material so that it does not move or peel during the lasing process. The material can also be laminated onto an IR transmissive material such as germanium, IR polymer, or sapphire using the capillary assisted method.

Thin elastomer films in particular suffer from significant warping and melting when machined with a CO₂ laser. This process allows for precise laser machining of thin films using affordable equipment.

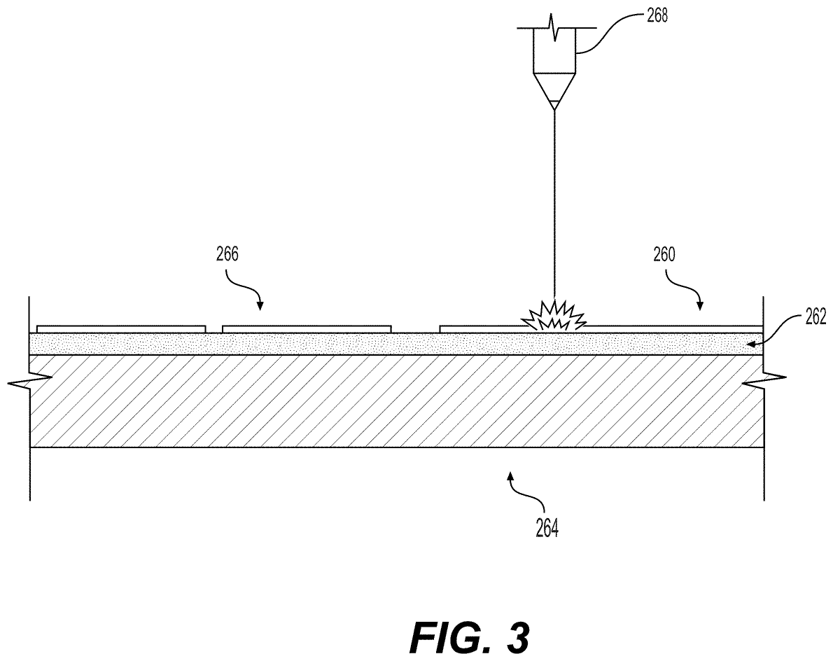

FIG. 3 is a diagram of the water assisted laser machining technique 120. A thin elastomeric polymer film 260 is held down on a substrate such as glass, germanium, sapphire, ice or IR polymer, using capillary action of a water film 262. The water 262 holds the cut film 266 down and absorbs some stray energy from the laser machining process.

C. Solvent-Based COC Glue

Solvent adhesives play a key role in permanently bonding two parts together. A pre-mixed glue is safer and easier to use.

The ability to apply adhesive layers quickly and uniformly offers a new method for bonding flat surfaces. This technique is simple and can be readily accomplished in a lab or manufacturing line. This process could be used for many kinds of adhesives, not only UV curable ones.

A solvent based glue made of dissolved Cyclic Olefin Copolymer (COC, TOPAS® 8007s04) consists of cyclohexane and acetone. COC pellets are dissolved in cyclohexane at a 1:4 volumetric ratio; this process takes several days. A solvent such as acetone is added until the mixture begins to change in optical property, indicating maximum solubility of COC in the cyclohexane/acetone mixture. Acetone lowers the glue viscosity and makes it less aggressive. The glue is high viscosity, and cures rapidly at room temperature. Toluene may be added to change the viscosity and evaporation characteristics of the glue. Curing of the glue can cause some bubble formation between bonded substrates, so small bonded areas are preferred. Glue ensures a strong and irreversible bond between two COC parts. Glue can be used to bond COC to glass and glass to glass. Use on plastics with low solvent resistance is not recommended. Application of the glue in a cold environment extends working time and improves solvent evacuation during curing.

D. Techniques for Selective Forming and Bonding of Thin Polymer/Elastomer Films

A process for selectively bonding regions of flat substrates in thermal bonding processes has been developed. Regions that are designed to remain unbonded are coated with a non-interactive material. Permanent marker and bovine serum albumin (“BSA”) have been demonstrated as simple and biologically compatible substances for selectively bonding COC substrates. This process has been applied to elastomeric material bonding processes but should be useful for other thermally bonded materials as well.

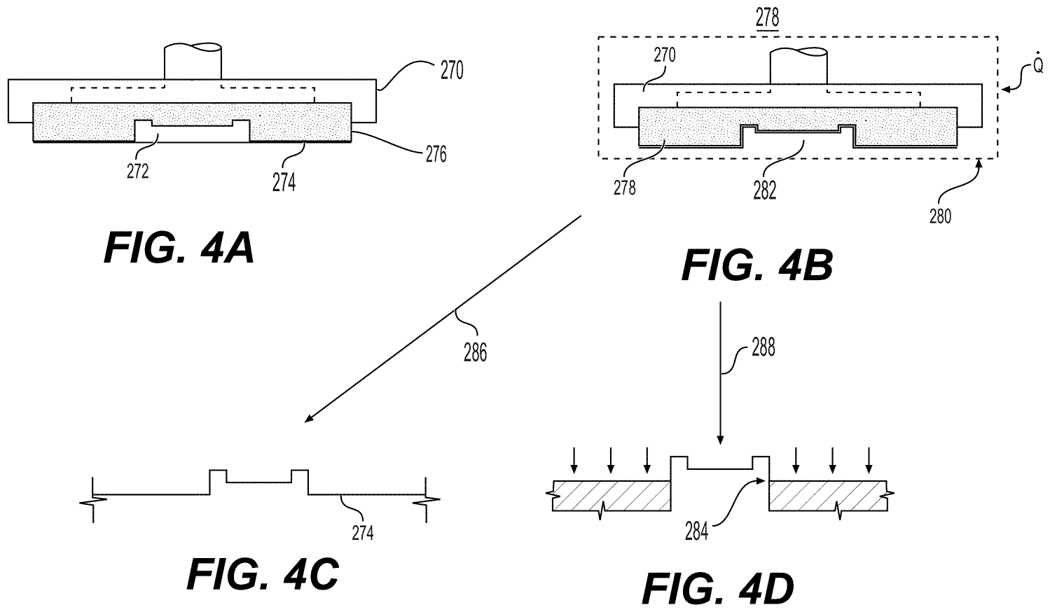

Another bonding procedure involves thermoforming a membrane during the bonding process by vacuuming the material into a semi-porous material such as a porous ceramic, as shown in FIGS. 4A-4D. The shape of the semi-porous material defines a negative mold for the membrane to deform into. If the material is held at its melt point during the bonding process it will retain its shape after the bonding process. Applications include pump diaphragm fabrication and valve development.

Any pressurized surface will bond during a thermal process. Some components, such as doormat valves, need to remain unbonded but retain surface to surface contact. Without the ability to control which surfaces bond and do not bond it is difficult to control the surface properties of the device design and it is also hard to ensure unobstructed fluid pathways in the device.

Selective bonding technique using a vacuum formed membrane utilizes a semi-porous material incorporated into one side of a thermally bonded device and formed to the intended negative shape of the membrane. Layers are assembled and the membrane is clamped between two substrates. Vacuum is applied to the semi-porous material causing the membrane to deform into the shape of the semi-porous feature. Heat and pressure are used in a thermal bonding step to bond the membrane to the two halves of the device. The membrane does not bond to the semi-porous material. The shape of the semi-porous material is retained by the membrane after bonding.

FIG. 4A-4D are cross-sectional views of a porous vacuum chuck with negative features that serve as a mold for thermoformed elastomer membranes (FIG. 4A), showing that vacuum deforms the elastomeric membrane into the mold (FIG. 4B), to yield a standalone thermoformed membrane (FIG. 4C), or can bonded to the manifold while hot (FIG. 4D). FIG. 4A-4D show the use of a porous ceramic vacuum chuck 270 with machined mold features 272 that serves as a template for thermoformed elastomer membranes 274. Membrane material 274 is laid onto the porous carbon material 276 and vacuum 278 is applied. Negative pressure draws membrane into the negative features of the mold. Heat 280 is applied to reach or exceed the membrane's melting point. The membrane 274 can then be cooled and released from the porous carbon chuck 276, or can be pressed against another polymer device while hot to create a permanent bonded membrane 284.

E. 3D Fluid Routing Using Laser-Cut Elastomer Films

Laser processing on thin elastomer films and the bonding process enables 3D routing of microfluidic channels without the need for hot embossing, machining, or other processes.

3D fluid routing can be accomplished using laser cut adhesive materials, but an elastomer is a more robust and solvent resistant option for generating microfluidic channels. This process ensures that the channel thickness is well controlled and is a better method for low-volume fluid routing.

### III. On-Chip Control and Sensing Elements for Microfluidic Devices

A. Cyclic Olefin Copolymer (“COC”) Elastomeric Structures Elastomeric materials such as those available from TOPAS® Advanced Polymers GmbH Raunheim Germany can be used to make elastomeric membranes that do not have the same problems as PDMS membranes. These materials are described in WO2011129869, “Melt blends of amorphous cycloolefin polymers and partially crystalline cycloolefin elastomers with improved toughness”. The TOPAS® COC resins are a chemical relative of polyethylene and other polyolefin plastics, are ultra-pure, crystal-clear and UV transparent, glass like materials, with broad global regulatory compliance. They are amorphous, with heat resistance in packaging film, sterilizable, thermoformable and shrink benefits. They have barrier properties to moisture, alcohols and acids.

B. Rolled Elastomeric Diaphragms An elastomer diaphragm with a stress relieving feature has been developed to be used in microfluidic valves and pump diaphragms. The membrane features a thermoformed semi-circular section that rolls during actuation rather than experiencing elastic deformation. The diaphragm is also designed to seat onto a manifold of a similar geometry. Actuation of the membrane is done using compressed gas and vacuum. A pump chamber can be designed to a specific displacement volume and valves can be designed to seal at a set pressure.

The rolling diaphragms can also be made of other materials than thermoplastic elastomers including thermoplastic films, rubber sheets, and silicones. Various shapes of rolling diaphragms can be explored to suit different applications (i.e. valves, accumulators, and pump chambers). Optimization can be done using iterative simulation in an FEA software.

Manufacture of these rolled diaphragms is facilitated by thermoforming using a porous carbon chuck and bonding.

Elastomeric micropumps and valves suffer from problems with reliability and well controlled fluid displacement. This valve design offers a low stress method for actuating elastic membranes of a variety of materials to make them more robust and effective. This design makes it easier to determine sealing pressures for valves and displacement volumes for pump chambers. This type of diaphragm experiences limited amounts of elastic strain and reduces the chance of plastic deformation and fatigue failure of a diaphragm. Applications include pump chambers, valves, volume storage, and fluidic accumulators.

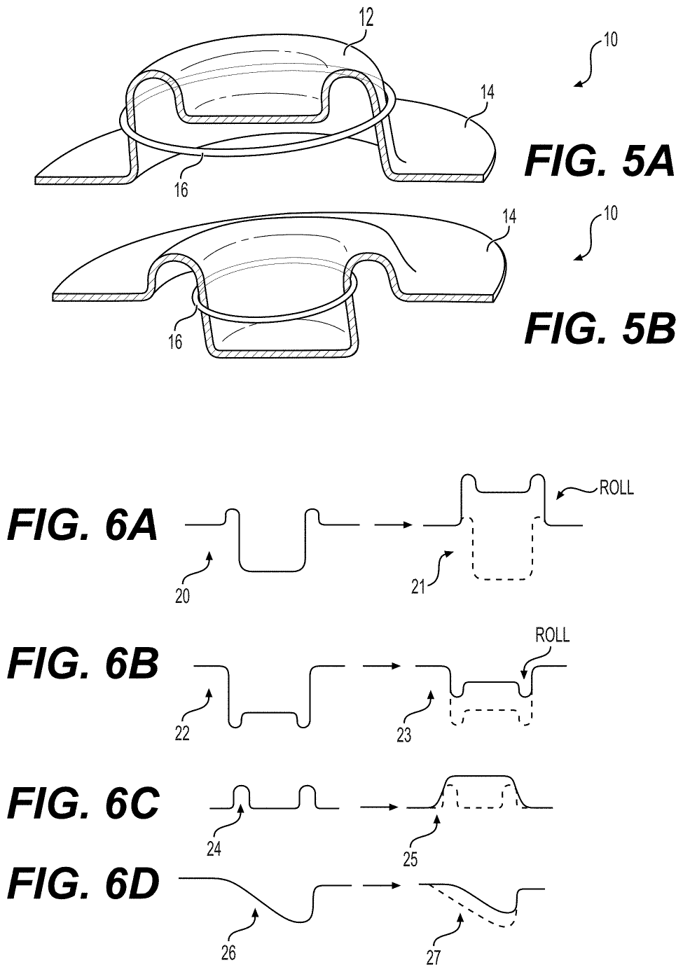

FIGS. 5A and 5B are prospective views of a rolling diaphragm 10 showing the hoop strain. The rolling diaphragm 10 has a rolling lip 12 with a lip 14, with a hoop 16.

FIGS. 6A-6D are schematics showing different types of rolling diaphragms. FIG. 6A is an external rolling diaphragm 20; FIG. 6B is an internal rolling diaphragm 22; FIG. 6C is a shape changing diaphragm 24; FIG. 6D is a sideways rolling diaphragm 26.

Each type of diaphragm can be thermoformed out of a variety of polymers and thermoplastic elastomers. Each type provides unique benefits with regards to volume displacement and stress management.

C. Optimized Diaphragm Pump Chambers

Diaphragm micropumps with optimized pump chambers that ensure reliable displacement volume and improved reliability have been developed. One pump chamber features a rolling diaphragm and one features a pump chamber with a predictable displacement stroke.

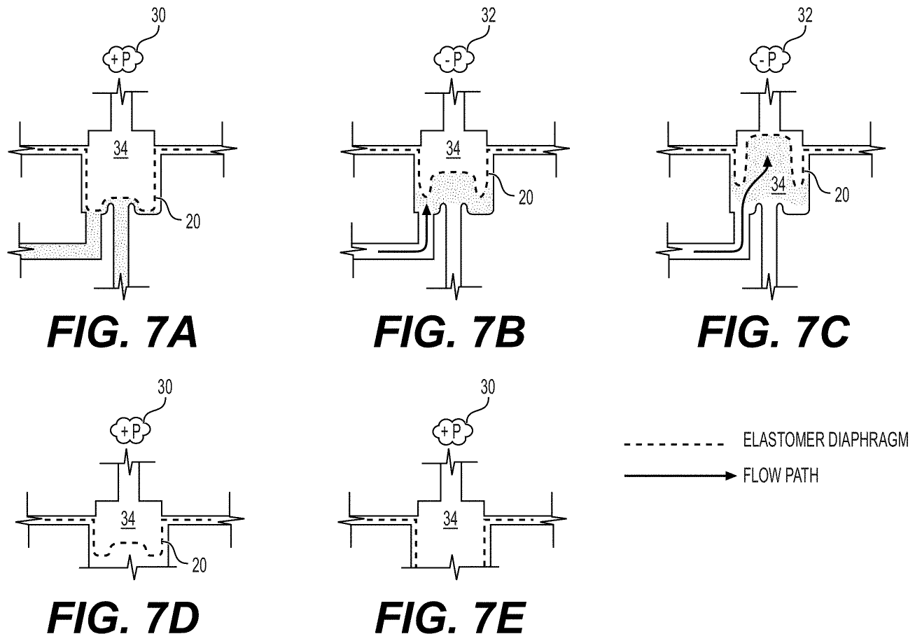

FIGS. 7A-7E are schematics of the mechanism of pumping using a rolling elastomer diaphragm. A pneumatic pressure source (+P) is used to displace the diaphragm. Vacuum (−P) is used to draw the diaphragm and fill a reservoir. Pressure is then applied for a displacement stroke. Before fluid aspiration, FIG. 7A; Vacuum is used to fill reservoir, FIG. 7B; chamber full of liquid, FIG. 7C; pressure is applied to chamber, FIG. 7D; end of displacement stroke FIG. 7E.

FIGS. 7A-7E are diagrams showing the mechanism of pumping using a rolling elastomer diaphragm 20. A pneumatic pressure source (+P) 30 is used to displace the diaphragm 20. Vacuum (−P) 32 is used to draw the diaphragm 20 and fill a reservoir 34. Pressure 30 is then applied for a displacement stroke. The rolling diaphragm pump chamber 30 uses a rolling diaphragm 32 to displace fluid volume in a chamber. The chamber includes a fluidic inlet and a valve. The diaphragm can be actuated using compressed gas and vacuum. A rolling diaphragm of any type could be used, but one with an internally rolling mechanism is preferred.

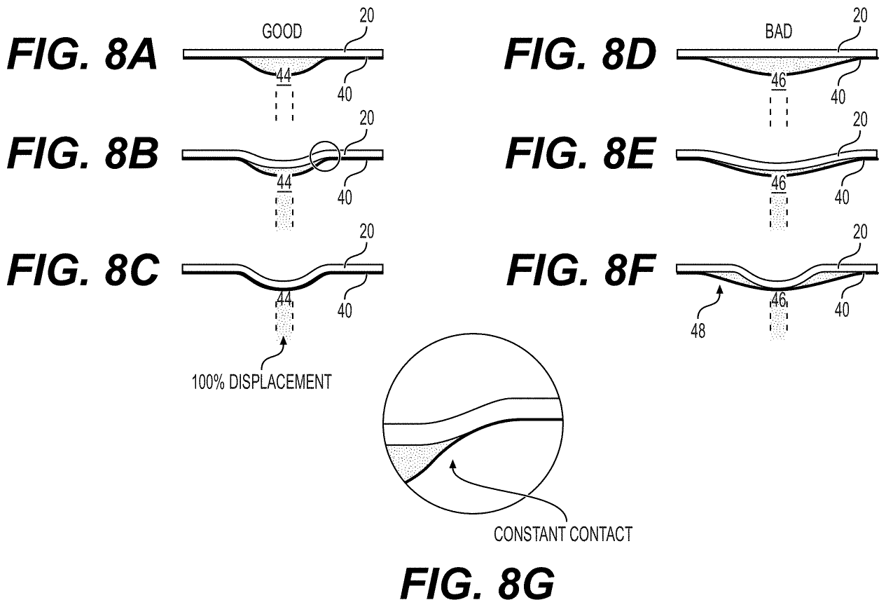

A second pump chamber design is an optimized shape that guarantees complete fluid displacement from the pump chamber. The chamber geometry is designed around the elastic response of a flexible membrane under pressurized load such that the membrane retains a ring of contact with the pump chamber during a pump stroke, as shown in FIGS. 8A-8F. This feature eliminates the chance for small pockets of fluid to get trapped in the diaphragm and ensure reliable displacement volumes. The pump chamber is also designed to hold a specific volume of fluid.

FIGS. 8A-8F are schematics of pump chambers 40, comparing an ideal pump chamber 44 with an unoptimized chamber 46. FIGS. 8A, 8B, 8C show the ideal pump chamber 44, where the diaphragm 20 maintains constant contact with the pump chamber 44 during actuation, as compared to the unoptimized chamber 46 of FIGS. 8D, 8E, and 8F, which risks trapping fluid 48 inside of the diaphragm membrane 20 causing unpredictable displacement volumes. FIG. 8G is an expanded view of the contact between the diaphragm and the pump chamber wall.

FIGS. 8A-8H are schematics of pump chambers 40, comparing an ideal pump chamber 44 with an unoptimized chamber 46. FIGS. 4A, 4B, 4C show the ideal pump chamber 44 where the diaphragm maintains 20 constant contact with the pump chamber 44 during actuation 44, as compared to the unoptimized chamber 36, 38, 40 of FIGS. 4D, 4E, and 4F, which risks trapping fluid 48 inside of the diaphragm membrane 20 causing unpredictable displacement volumes.

Since most pump chambers in the literature feature a cylindrical bore and a diaphragm that flexes into the bore with no constraint, this alternative embodiment offers no stress management and does not provide a deterministic displacement volume for a single stroke of the pump. The rolling diaphragm pump chamber offers a low stress and volumetrically constrained pump chamber.

D. On-Chip Microfluidic Pressure Regulators

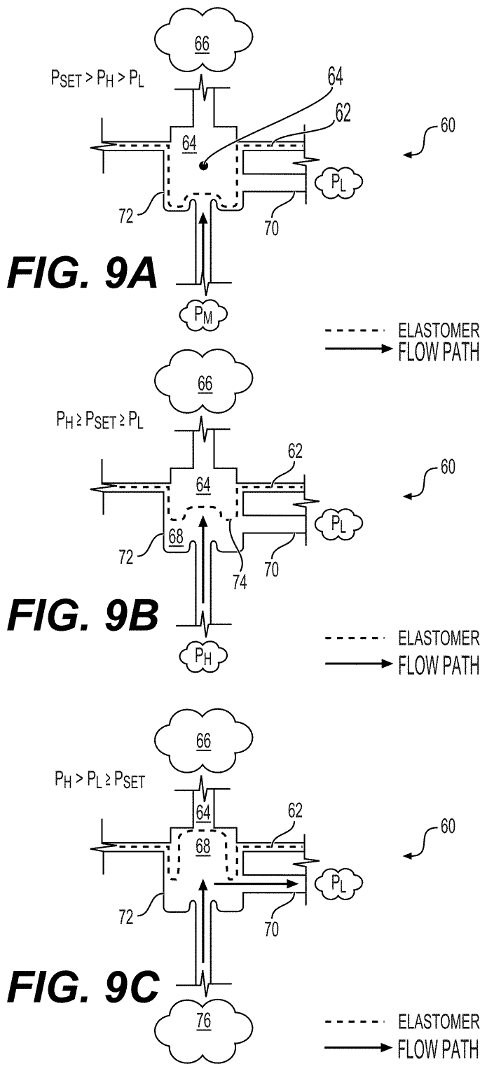

A microfluidic pressure regulator 60 that uses a pneumatically actuated elastic membrane 62 as a sealing feature and compressed gas 64 as a bias has been designed and is shown in FIGS. 9A-9C. FIGS. 9A-9C are schematics of a microfluidic pressure regulator 60 that uses a pneumatically actuated elastic membrane as a sealing feature and compressed gas as a bias. Fluid builds up pressure against the elastic membrane until it overcomes the pressure exerted by the compressed gas on the other side. FIGS. 9A, 9B. The fluid begins to flow once the fluid pressure exceeds the sealing pressure. FIG. 9C. Fluid pressure can be regulated by adjusting the compressed gas source and the flow can be stabilized by adding compliance in the fluidic circuit.

This back pressure regulator 60 uses a rolling diaphragm 62 as a sealing and sensing element. When the upstream pressure 64 exceeds the pressure setpoint 66, the diaphragm 62 is displaced until fluid 68 is able to flow through the side 70 of the diaphragm chamber 72. Sealing at the side 74 of the chamber 72 occurs when the pressure setpoint 66 is greater than the upstream pressure 64. Fluid 68 builds up pressure against the elastic membrane of the diaphragm 62 until it overcomes the pressure exerted by the compressed gas 66 on the other side. The fluid 68 begins to flow once the fluid pressure 76 exceeds the sealing pressure. Fluid pressure can be regulated by adjusting the compressed gas source 64 and the flow can be stabilized by adding compliance in the fluidic circuit.

This is the first on-chip pressure regulator. Pressure driven flow systems are common and commercially available, but these systems rely on fluid mechanics to determine system flowrates. This technology enables the control of system pressures with the use of any volumetrically controlled pump.

Studies have demonstrated that a microfluidic accumulator and pressure regulated valve can serve as a pressure regulating device on a chip. This regulated fluid pressure to 14 psi using a pressure source and a diaphragm pump.

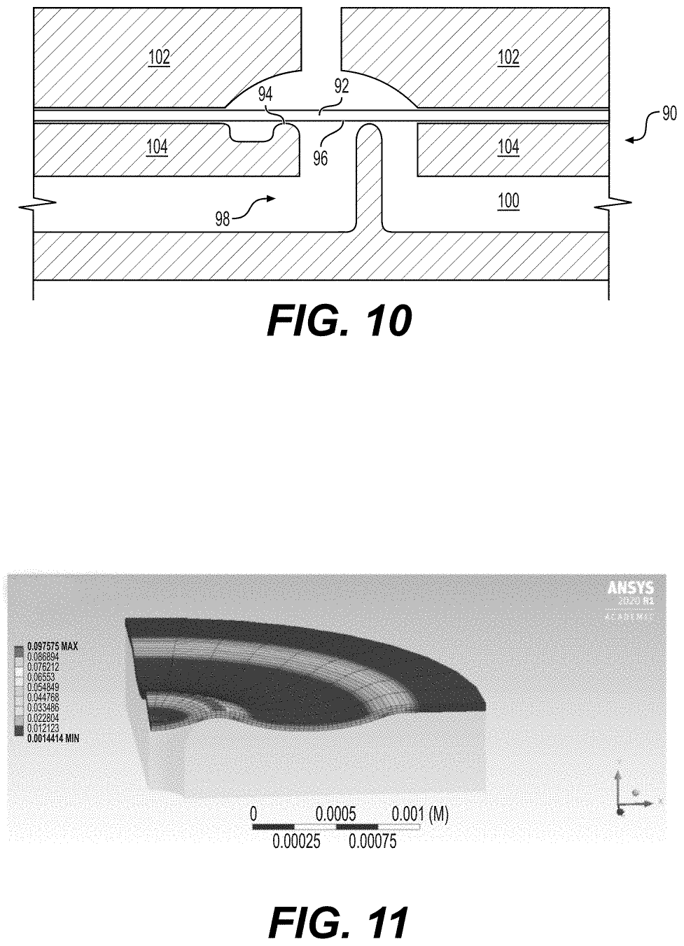

E. Optimized Microfluidic Diaphragm Valves An active microfluidic valve for on-chip control of fluid passage features a semi-circular lip that defines a line of contact for an elastic membrane, as shown in FIG. 10. FIG. 10 is a simple diagram of a valve 90 with a bonded elastic membrane 92 and a defined sealing contact 94. Fluid flow can be bi-directional. Sealing lip 94 can be a small flat surface or a rounded shape as shown.

The sealing surface 96 is only located on one inlet 98 of the valve and the other fluid inlet 100 is free from contact with the elastic membrane. The elastic membrane 92 is actuated using compressed gas and is bonded to the separate halves 102, 104, of the fluidic manifold. This valve design allows for bi-directional fluid flow.

This design avoids the problem with many elastomer diaphragm valves having trouble generating a reliable seal. Doormat and one-way flap valves suffer from thin film fluid flow and fluid creep around the sealing surfaces.

FIG. 11 is a valve has a rounded sealing feature that amplifies the sealing pressure at the inlet of the valve, showing the valve in cross section with the membrane experiencing a higher strain and contact pressure at the sealing interface.

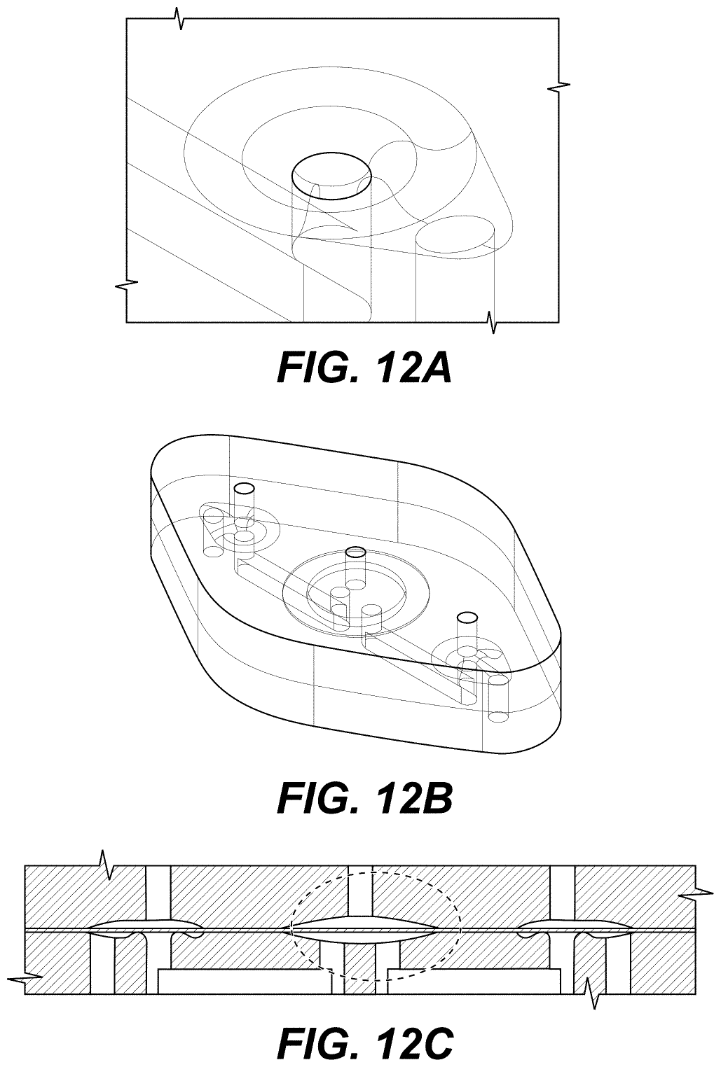

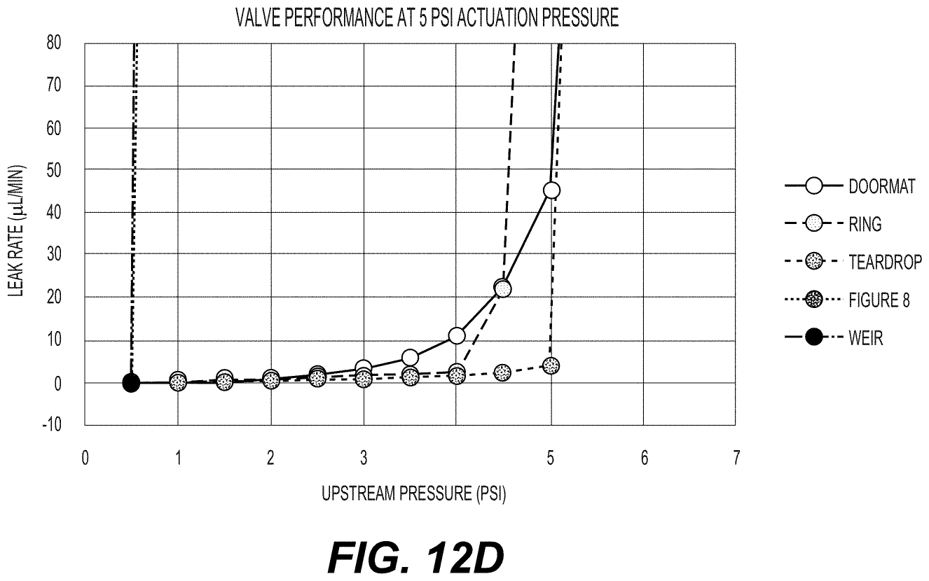

FIGS. 12A-12C are a teardrop shaped valve with rounded sealing surface. FIG. 12A is a perspective view of the teardrop shaped valve with a rounded sealing surface and a teardrop shape that reduces the overall volume of the valve. The teardrop shape reduces the dead volume of the valve when compared to a circular profile valve of the same size inlet. Here is a screenshot of the teardrop valve in CAD. Sealing shape in red dashed line. FIG. 12B shows the valve integrated in a pump. FIG. 12C is a cross-sectional view of the valve integrated in the pump. FIG. 12D is a graph comparing the performance of various vales (doormat, ring, teardrop, valve in FIG. 8), demonstrating that the teardrop valve exhibits improved performance over out previously designed doormat valves.

Further improvement can be made to the valve by reducing the total volume of the valve. A preferred configuration of this valve is a teardrop shape that creates a fluid path for the outlet of the valve but does not add extra volume radial from the sealing surface. The shape of the valve is lofted to reduce the volume but also provide a smooth and continuous surface.

F. Microfluidic Accumulators

Fluidic accumulators play a key role in large-scale hydraulic circuits but have not been developed commercially for microfluidic systems. Accumulators fill the need of buffering fluid flow by temporarily storing fluid volume under pressure. These components are similar to capacitors in electrical circuits.

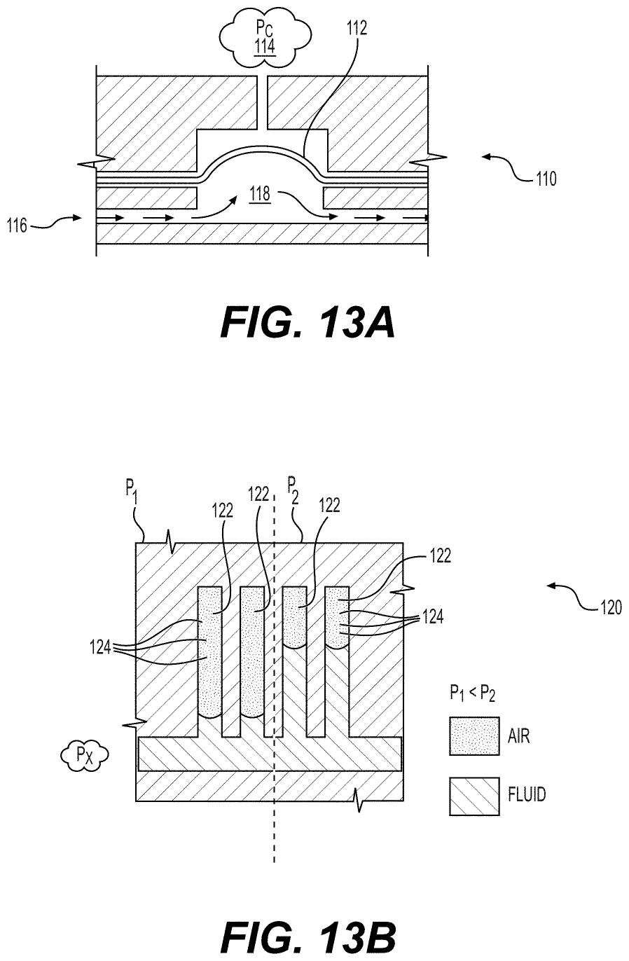

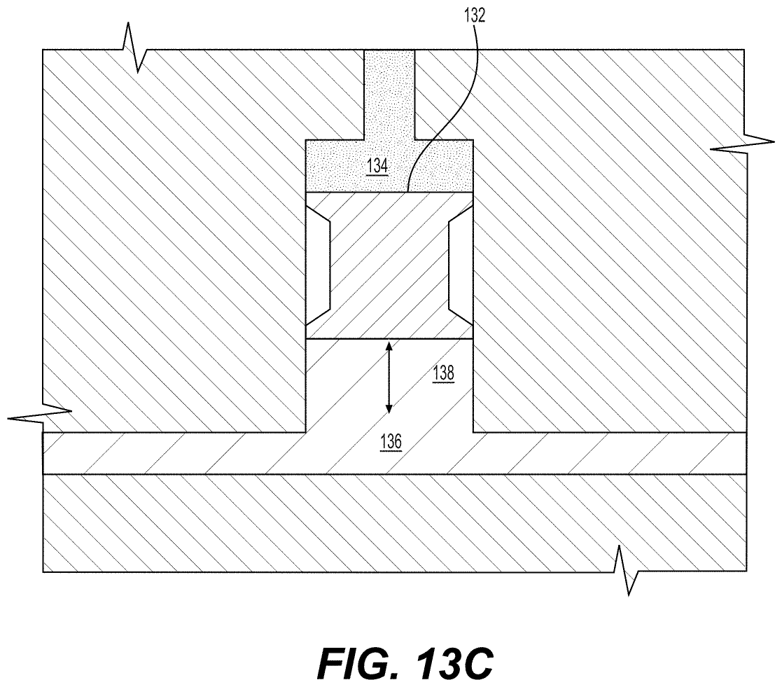

FIGS. 13A-13C are schematics of several different types of microfluidic accumulators. FIG. 13A is a schematic of an accumulator using a flexible membrane to store pressure using stored elastic energy in the membrane. FIG. 13B is schematic of a microfluidic accumulator using small dead-end microfluidic channels for trapping gas bubbles and storing volume under pressure. FIG. 13C is a schematic of a microfluidic accumulator that uses a piston pressurized with air on one side and fluid stored in a reservoir.

Several different types of microfluidic accumulators can be used to store pressurized fluid in a microfluidic chip. Pressure is stored using compressed gas, surface tension phenomena, or elastic strain energy. A microfluidic accumulator 110 can use a rolling diaphragm 112, as shown in FIGS. 13A-13C. The diaphragm 112 is pressurized with air 114 on one side and fluid 116 is stored in a reservoir 118 below. When the fluid volume exceeds the air pressure the diaphragm 112 is able to move to store excess volume.

The accumulator 110 uses the flexible membrane 112 to store pressure. Elastic deformation yields a change in volume of the component. This kind of accumulator can be tuned by changing the pressure on the back of the membrane and by changing the size (i.e. thickness and diameter) of the membrane.

A microfluidic accumulator 120 shown in FIG. 13B can use small dead-end microfluidic channels 122 for trapping gas bubbles 124 and storing volume under pressure. Gas bubbles 124 are trapped and compressed when more volume enters the channel 112. This type of accumulator was successfully tested on a standalone microfluidic chip.

A microfluidic accumulator 130 shown in FIG. 13C can use a low-friction piston 132 to store fluid volume. Air pressure 134 is applied to the back side of the piston 132 and pressurizes the fluid 136 on the other side. Fluid 136 is stored in the bore 138 of the piston.

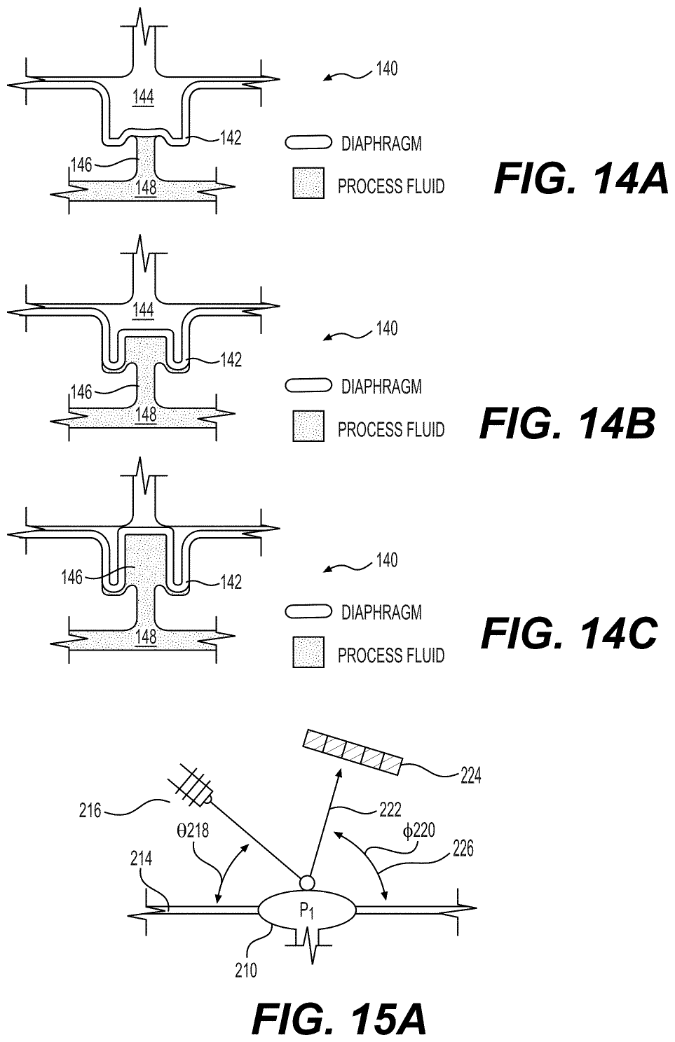

FIGS. 14A-14C is a microfluidic accumulator, with a diaphragm pressured with air on one side and fluid is stored in a reservoir, no volume (FIG. 14A), accumulating volume (FIG. 14B), and at capacity (FIG. 14C). A microfluidic accumulator 140 can use a rolling diaphragm 142, as shown in FIGS. 14A-14C. The diaphragm 142 is pressurized with air 144 on one side and fluid 146 is stored in a reservoir 148 below. When the fluid volume exceeds the air pressure the diaphragm 142 is able to move to store excess volume.

G. Pressure Sensing using Elastic Membrane Deflection and Trapped Gas Accumulator

A pressure sensing method leveraging an elastic membrane that deflects under pressure and an optical lever. The membrane can be coated with a reflective material to reflect incident light. A laser can be aimed at the membrane and reflected off of the membrane surface. The laser can be directed to a photodetector that either senses position or light intensity. If light intensity is selected then a diffraction grating may be used to split the light based on position on the grating.

An optical lever may provide a pressure sensing method that is extremely sensitive for even small changes in pressure. Most pressure sensors on the market sense pressure on the order of psi, while some microfluidic applications require pressure sensing in fractions of psi.

The trapped gas pressure sensor is useful because the sensing feature (a camera) is not a part of the microfluidic device and therefore does not add to the cost of the chip. This sensor is also linear, which makes for easier calibration and measurement.

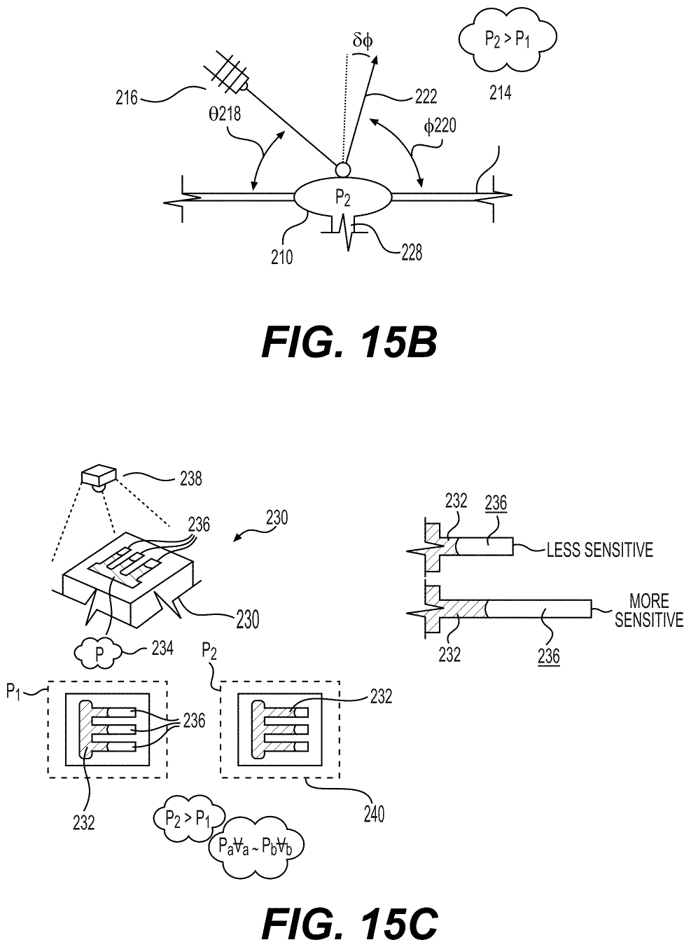

As shown in FIGS. 15A-15B, a pressure sensor 210 featuring an optical lever 212 and a deformable membrane 214 can be utilized. The membrane 214 can be a reflective material or have refractive index properties. A laser 216 is aimed at the membrane 214 and reflected off the surface. The output angle 218 changes as a function of membrane deflection 220 under pressure. The laser output 222 is incident on a photodetector 224. A diffraction grating and intensity measurement or a position sensing method also could be implemented.

Pressure sensing using the properties of trapped gas microfluidic accumulators can also be used, as shown in FIG. 15A-15C. The length of the gas bubble 232 is directly proportional to the pressure 234 of the liquid 236 in the microfluidic channels. As pressure 234 builds up the trapped gas, bubbles 232 are compressed and a camera 238 or other optical detector can be used to sense the change in length of the bubble or liquid phase.

H. Liquid Level Sensing

Liquid level sensors can be found for many large scale fluidic systems, but few technologies exist for tracking fluid volumes in microfluidic chips. Sensing of fluid volumes in a non-invasive and accurate manner is helpful for monitoring of onboard fluidics and determining when fluids need to be exchanged or trafficked to other parts of the chip. This can also help to control hydrostatic pressures on the chip.

Liquid level sensing methods for small scale microfluidic reservoirs can utilize a deformable membrane which deflects under hydrostatic pressure. Level sensing by visually tracking fluid height in a reservoir with a camera can be done using direct measurement, light transmission and color saturation properties, or tapered reservoirs.

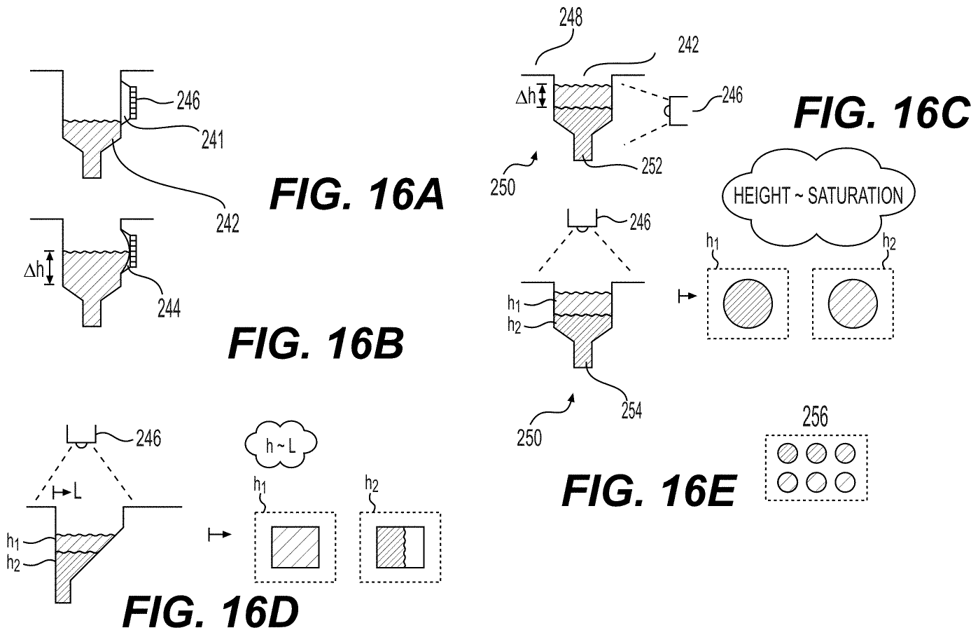

Liquid sensing methodologies for microfluidic reservoirs are shown in FIGS. 16A-16E. FIGS. 16A-16E are schematics of liquid sensing methodologies for microfluidic reservoirs where a deformable membrane is incorporated into the media reservoir under hydrostatic pressure, for changes in capacitance, resistance between contacting materials or optical properties (FIG. 16A). FIG. 16B shows the membrane deflecting under pressure. FIG. 16C shows the fluid reservoir having a clear window or side, where changes in fluid levels are measured and recorded by a camera. FIG. 16D shows a similar fluid reservoir where the camera is positioned above the reservoir. FIG. 16E is a schematic of the camera taking images of the fluids containing dye to provide for optical measurement.

A deformable membrane 241 incorporated into the media reservoir 242 can be deflected under hydrostatic pressure 244. The membrane 241 contacts another surface for changes in capacitance, resistance between contacting materials, or can be observed using an optical system 246 (FIG. 16A). Additional optical sensing methods include observation of liquid levels from the sides of microfluidic devices for direct measurement or from above using correlated measurements (FIGS. 16B, 16C). A reservoir 250 with a taper can be designed so that the fluid's free surface area changes as a function of fluid height. Optical transmission and color saturation properties can be utilized as well (FIG. 16D, 16E); color saturation and optical transmission will be a function of the fluid height in the reservoir.

I. Microfluidic Caps for Cell Observation and Manipulation

Sterility and ease of access in microfluidic devices is key for many lab-on-a-chip and experimental applications. For example, being able to exchange culture media and manipulate cell cultures requires device access for a needle or pipette. New types of caps that offer a simple and sterile way of interacting with a chip will enable these procedures for microfluidic chips. Ideally, these caps are optically clear to allow for imaging or background illumination. Further, a single use and disposable cap is helpful for sterility reasons.

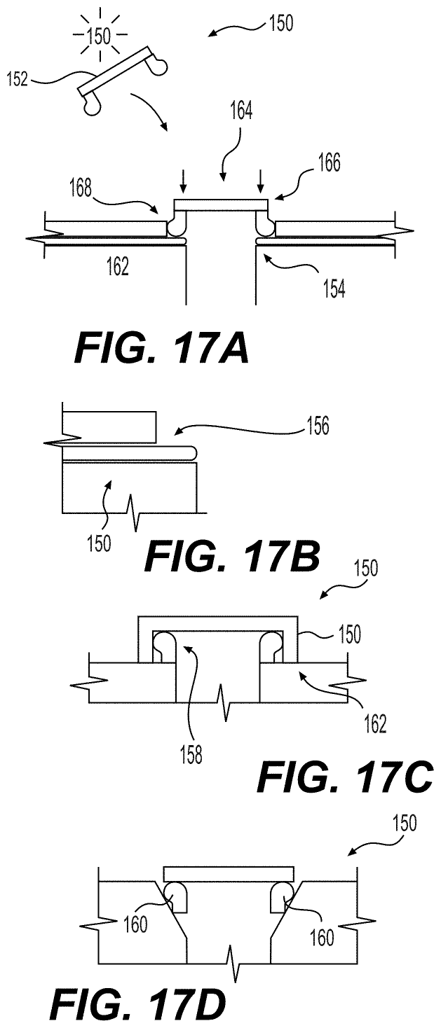

FIGS. 17A-17D are schematics of removable caps for cell culture applications. An optically clear snap on cap is shown in FIG. 17A. An elastomeric feature on or under the caps adds compliance, as shown in FIG. 17B. A cap formed of an optical film with a patterned adhesive for sealing is shown in FIG. 17C. A press fit seal or compressed elastomeric feature on the underside of the cap is shown in FIG. 17D.

The removable cap can be included for cell culture applications in one embodiment shown in FIG. 17A. The top 152 of the cap 150 is optically clear and is able to be sealed 154. Sealing can be accomplished with a press fit, clamped gasket, or rubber/elastomer seal. The cap 150 can be removed for culture sampling and manipulation. A press fit can be defined like that used in Eppendorf tubes and PCR caps. This is similar to many cap designs in the cell culture field.

A sealing feature can also be created by exposing part of the bonded elastomer feature 156 to the cap, as shown in FIG. 17B. This adds compliance to allow for a well-defined sealing surface.

An alternative to a press fit cap or a gasketed interface is an adhesively bonded window as shown in FIG. 17C. A “cap” could consist of an optical film with a patterned adhesive that is used to seal the optical film onto a device. This type of sealing feature could provide a sterile, single use, and cheap method for sealing of microfluidic chips.

As shown in FIG. 17D, caps can have a press fit seal or use some sort of compressed elastomeric feature 160. Exposed elastomeric material can serve as a gasket for sealing of the cap which can be compressed using strain energy or a clamp/latch. An adhesive sticker can be used for sealing flat surfaces of a microfluidic device.

J. Pneumatic Connections to Microfluidic Chips

Most commercially available pneumatic connectors are either one-tube-at-a-time or feature a threaded fastener. These operations waste time which can be critical to outcome for some experiments. A quick connect mechanism is useful because some operations in microfluidic experiments are time sensitive. For example, a chip cannot be disconnected from pumping for extended periods of time. However, disconnection may be required for accessing fluid volumes, manipulating cell cultures, or taking images on a microscope.

A quick connection for pneumatic lines to a microfluidic chip can be achieved with a spring loaded or clamped gasket as shown in FIG. 11-12. The ability to quickly connect and disconnect microfluidic chips to pneumatic lines facilitates rapid exchange of microfluidic chips with reliable sealing for all pneumatic connections.

Quick release features for microfluidic chips 170 can incorporate a compressed gasket or an array of O-rings 172 compressed using a spring 178 loaded lever 174, a toggle clamp 176, or an overcenter latch 180, shown in FIGS. 11, 12A and 12B. These clamping mechanisms facilitate easy connection of pneumatic and fluidic lines to a microfluidic chip without the use of tools or screws.

K. Dynamically Controlled Pressure Regulation for Actuation of Pump Diaphragms

Rapid actuation of pump membranes causes instantaneous peaks in flow velocity that may have a negative effect on flow stability. In biological applications, the dynamic actuation of a micropump implies significant shear stress that may influence and potentially harm living components.

In one embodiment, the system composes a programmable pressure source for dynamic pressure control of pump chambers. The pressure to actuate an elastic membrane is controlled from vacuum to positive pressure slowly so that the membrane flexes slowly. Gradual actuation of a pump chamber lowers the pulsatility of the pumping system and stabilizes the pump flow.

L. Microfluidic Oxygenators Made with Thin Elastomer Film

Oxygenation plays a key role in cell culture and lab-on-a-chip applications. A microfluidic oxygenator with a biocompatible and low absorption gas permeable membrane has been developed. Long aspect ratio microfluidic channels create a large diffusion surface for the gas transfer and a thin membrane promotes optimal gas transfer. The gas permeable material is preferably an elastomer such as a Cyclo olefin copolymer (COC). These are transparent amorphous thermoplastics produced by copolymerization of norbornene and ethylene using a metallocene catalyst. These copolymers have many attractive optical properties including high clarity, high light transmissivity, low birefringence, and high refractive index. Other performance benefits include excellent biocompatibility, very low moisture absorption, good chemical resistance, excellent melt processability and flowability as well as high rigidity, elastic modulus, and strength which are retained over a wide temperature range, from about −50° C. to near their glass transition temperature.

Alternative elastomeric materials include (styrene-ethylene-butylene-styrene (SEBS) or a thin rigid material such as Polyether ether ketone (PEEK), a colorless organic thermoplastic polymer in the polyaryletherketone (PAEK) family, a semicrystalline thermoplastic with excellent mechanical and chemical resistance properties that are retained to high temperatures, Perfluoroalkoxy alkanes (PFA, PTFE) are copolymers of tetrafluoroethylene and perfluoroethers, characterized by a high resistance to solvents, acids, and bases. or PTFE. Other materials may be considered based on gas transport properties. The performance of the oxygen transport can be determined by the oxygen transmission rate of the material, determined by ASTM D3985. Improved performance of the oxygenator can be achieved using a higher concentration of oxygen, increasing the partial pressure of the gas, and potentially by flowing the gas over the transfer surface. Gas exchange can be monitored using feedback from oxygen sensors.

COC elastomers can be bonded at long thin aspect ratios for use in oxygenator design. Other material may require a different lamination process.

### IV. Hydrogel Scaffolds

A. Cell Support Scaffolds Using Macro-Porous Elastomer Films

An optically clear, low stiffness cell support scaffold has a wide range of applications in cell biology. Most commercially available cell support scaffolds are not image friendly and are made of a rigid material, usually polystyrene.

A cell support scaffold to be used in microfluidic chips and transwell inserts is made of a hydrophobic elastomer that is optically clear with low autofluorescence. The pore size can be tailored to the specific application, but even large pores (˜1 mm diameter) are possible because of the hydrophobic nature of the material. This structure can be used to suspend cells in liquid or in cell-laden hydrogels. This type of scaffold is low modulus which poses a benefit to cell adhesion and stress response.

B. Casting of Hydrogel Structures as Cell Scaffolds

Hydrogel containment offers an alternative to the meniscus pinned techniques commonly used in similar devices. This design offers a benefit to the experiment because it allows the gel to swell, allows for direct access to the cell culture, and offers a more flexible and reliable solution to gel installment.

Cell-laden hydrogels were installed into a microfluidic device. The hydrogel is injected into a separate compartment and then polymerized. If necessary, the hydrogel is allowed to swell by means of liquid absorption. The capsule can then be inserted into a microfluidic chip with fluidic connections and gasketed interfaces. One embodiment of this compartment includes removable structures that serve as templates for microfluidic channels. The base of the capsule is an image friendly material so that biological microstructures and cell behavior can be observed in situ. These hydrogel compartments are specifically designed to promote a perfusable vascular network between two media channels.

C. Insertion of Pins into Hydrogel to Stabilize Gels

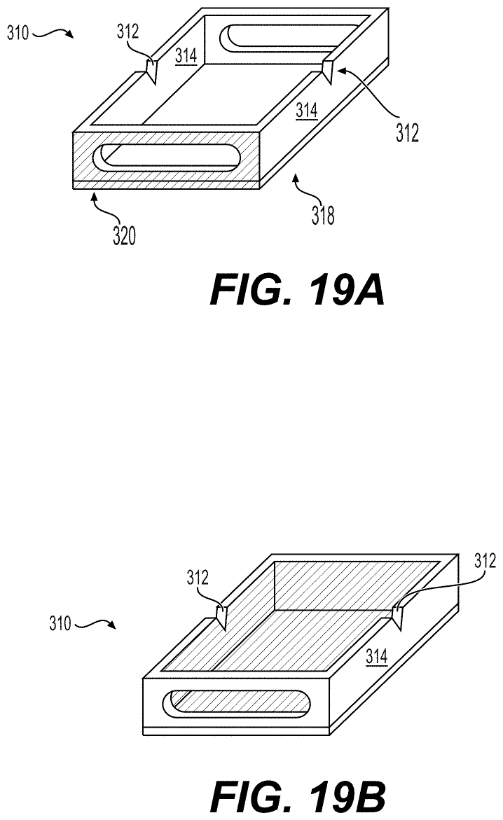

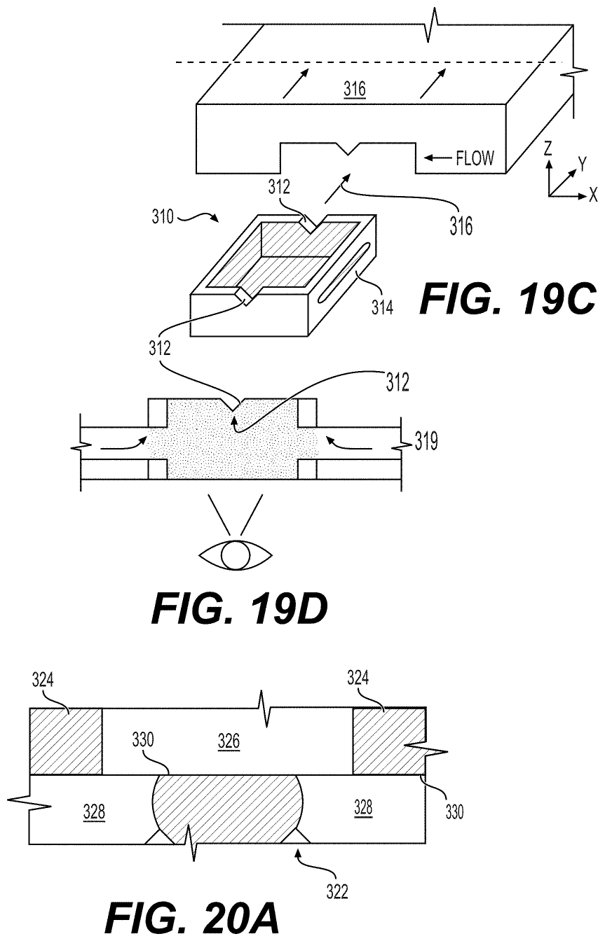

A hydrogel compartment 290 featuring removable support structures 292 is shown in FIGS. 19A-19D 18A-18D. The container 294 holds the hydrogel 296, overlaid with media 298. Pins 292 are inserted into the hydrogel chamber 296 to stabilize the gels as formed.

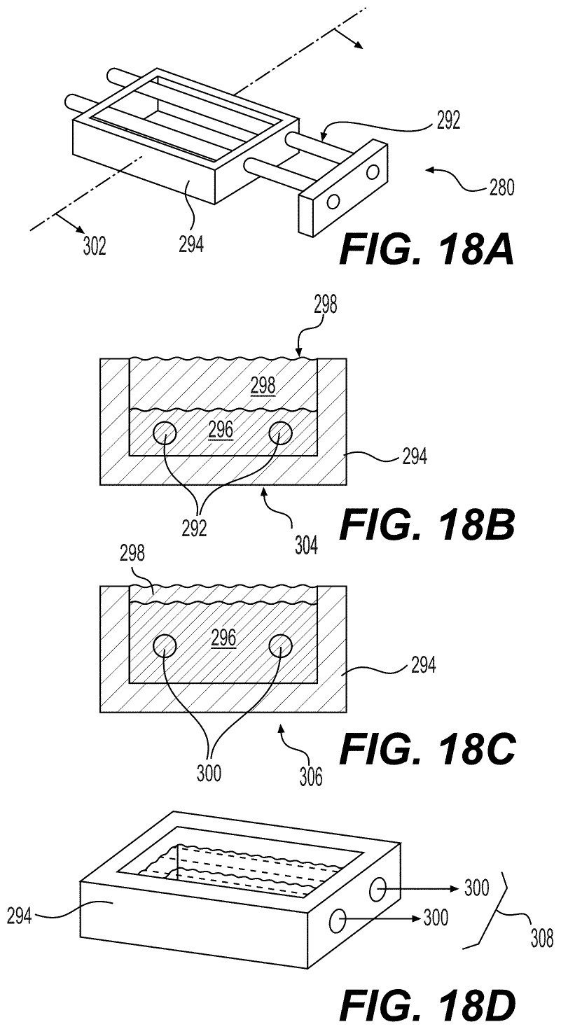

Once the pins 292 are removed, the pin cavity 300 (FIGS. 18B, 18C) can be used as a fluidic channel. Removable pins 292 should be made of a hydrophobic material so that the hydrogel does not get stuck to the removable pin 292. Most fluorinated polymers (PFA, PTFE, etc.) will work for this application.

FIGS. 19A-19D show a hydrogel compartment 310 with wide flat channels 312 at the sides 314 of the tissue compartment. The sides 314 of the compartment 312 allow for media flow across the sides of the tissue compartment. Gel is put in 310 and allowed to swell with media to fill the volume (FIG. 19A-B). Wedge creates a seal along the X direction (FIG. 19D)

Gel is inserted through a port 325 into a container containing removable support structures 322 such as PHASEGUIDES®®. These support structures can be sharp ridges or walls 324 that extend across the entire media channel. After gel polymerization the channels 326 are filled with media. PHASEGUIDES® 322 dissolve into media. Once the PHASEGUIDES® 322 dissolve the gel 328 is allowed to swell into the media channels.

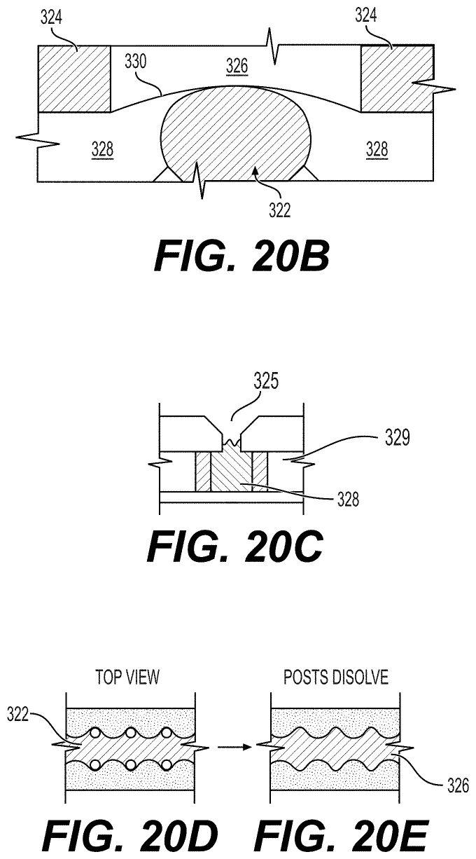

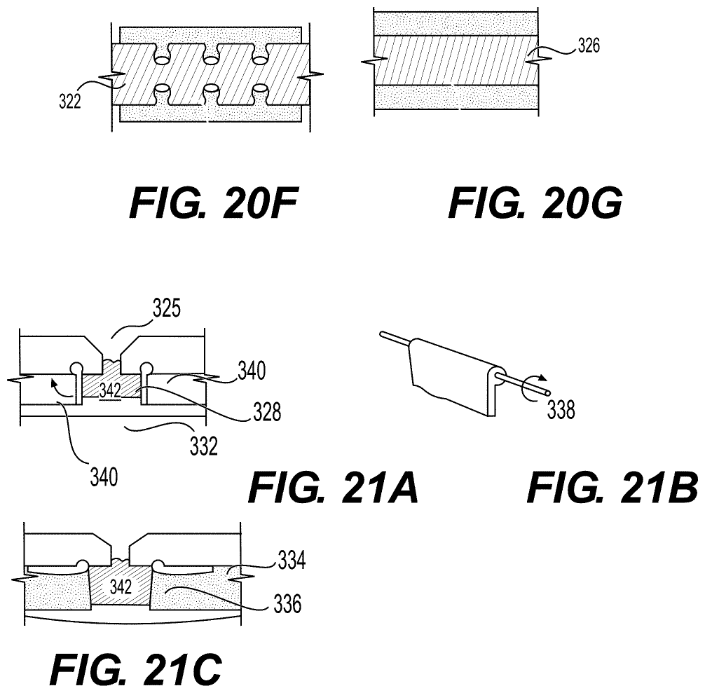

A PHASEGUIDE®-type hydrogel insertion method with a hyperelastic material backing 330 allows for gel expansion and swelling. With posts, gel is over constrained (FIG. 20F) and without posts, gel is free to expand (FIG. 20G).

D. Hydrogel Installation Using Flap or Hanging Drop

The hydrogel can be installed into the compartment using a method for creating sealable fluidic channels such as a rotating flap mechanism, shown in FIG. 21A-21C. Flap 340 hangs down creating a seal at the time of gel installation. Once the gel 342 polymerizes, flap 340 is rotated about an axis 342 to expose the sides of the gel channel. The flap can be made of a hydrophobic material and/or an elastomer (FIG. 21B) to create a seal during gel installation. A preferred material for cell culture are fluoropolymers including PTFE and PFA. The flaps 340 may be open to reveal the media channel 334 and the gel is free to expand 336 (FIG. 21C).

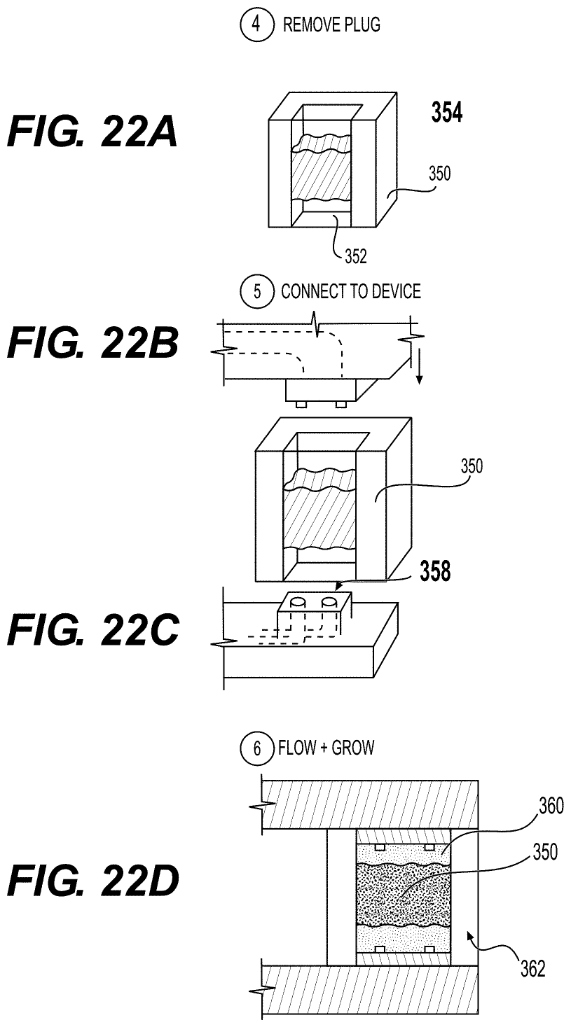

Gel installation using dissolvable compartments is shown in FIGS. 22A-22D. The dissolvable material acts like a fillable container 350 for gel 352 installation (FIG. 22A). The gel goes into the compartment 350 and polymerizes (FIG. 22B). Multiple compartments allow for multiple gel types. The compartment dissolves into the media (FIG. 22C). Once the compartment is dissolved, the gel 352 swells to fill the container 350 for fluid flow into and out of the gel (FIG. 22C, 22D).

A hydrogel installment method can use hanging hydrogel drops that swell into a sealed shape. One may still require the use of PHASEGUIDES® or some other type of support structure in a meniscus pinning technique. In this embodiment, the hydrogel is installed using a slot shaped hanging drop profile. This method allows for multiple flow patterns as depicted. Hanging drop could expand until the drop presses against another feature in the device to create a seal.

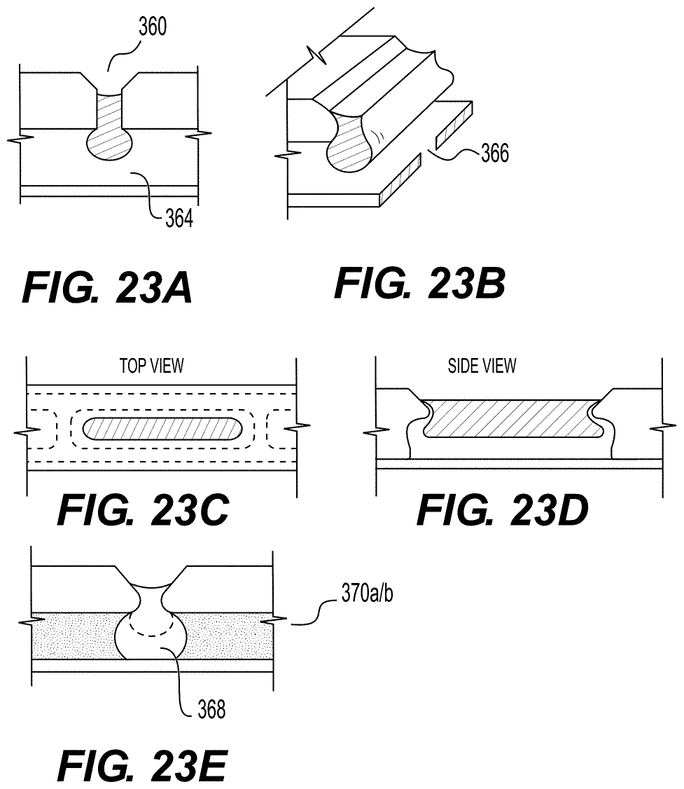

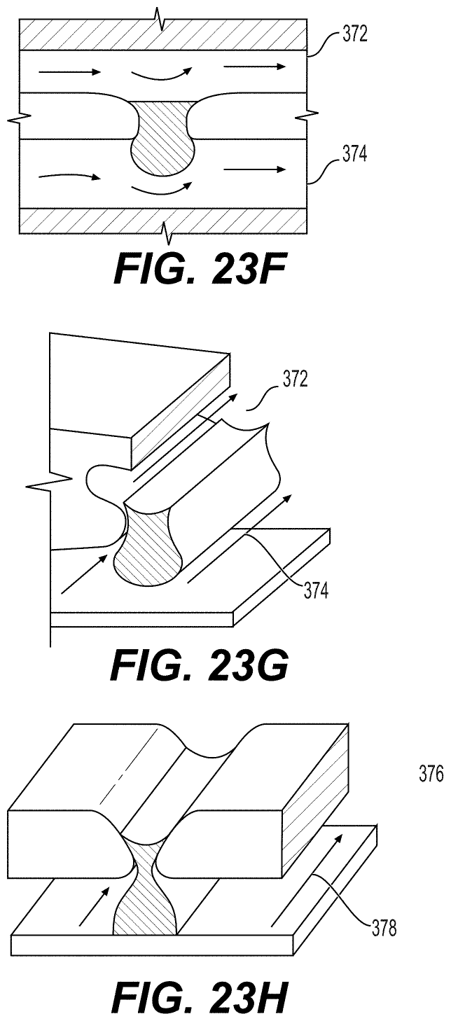

Hydrogel installations using a slot shaped hanging drop profile are shown in FIGS. 23A-23E. This method allows for multiple flow patterns as depicted. The hanging drop can expand until the drop presses against another feature in the device to create a seal.

FIGS. 23A-23E are cross-sectional schematics of a slot shaped hanging drop hydrogel help in place by surface tension (FIG. 23A), the top and side views (FIG. 23B), where the gel is swollen to separate a media channel into two channels (FIG. 23C), and the resulting flow configurations: across the top (FIG. 23D), under the drop (FIG. 23E), along the length of the drop (FIG. 23F), and along the sides (FIGS. 23G, 23H).

As shown in FIG. 23A, the gel 360 is installed through a port 362 where the hydrogel drop 364 hangs in place due to surface tension. This can run across a width to form a long hanging drop 366 as shown in FIG. 23B, or be in the form of a single drop. FIG. 23C shows a top view of hanging drop 366 and FIG. 23D shows a side view of hanging drop 366.

FIG. 23E shows how the hydrogel drop 368 can expand to seal off the connection between two regions of a media channel 370a, 370b. FIG. 23F shows that one can have continuous flow 372 across the top of hanging drop 368 and an obstructed flow 374 under the bottom of hanging drop 368. FIG. 23G shows the flow channel 372 across the top and the flow channel 374 along the bottom from the side. FIG. 23H shows the hydrogel 366 and flow channels 372 and 374 within the device 376.

### V. System for High Throughput Microfluidic Experiments

A. Electro Pneumatic Control Manifolds with Connection to Multiple Microfluidic Chips

Most microfluidic platforms are designed to be operated one chip at a time. This requires substantial infrastructure and tubing to control multiple chips at once. A system that facilitates easy access to multiple chips allows for more robust experimental designs and open up the ability to run duplicates and controls.

A manifold keeps normal gravitational alignment for the chips by using a tower or a carousel. If the chips were oriented in a different fashion it is possible that the chips will not function properly or might experience leaking.

An integrated electro pneumatic manifold for connection and control of many microfluidic chips can be utilized. Rather than connecting pneumatic lines to a chip one chip at a time, multiple can be connected to the same pneumatic manifold. This limits the amount of controllers, pressure sources, and other components required to run an experiment with duplicates and control conditions.

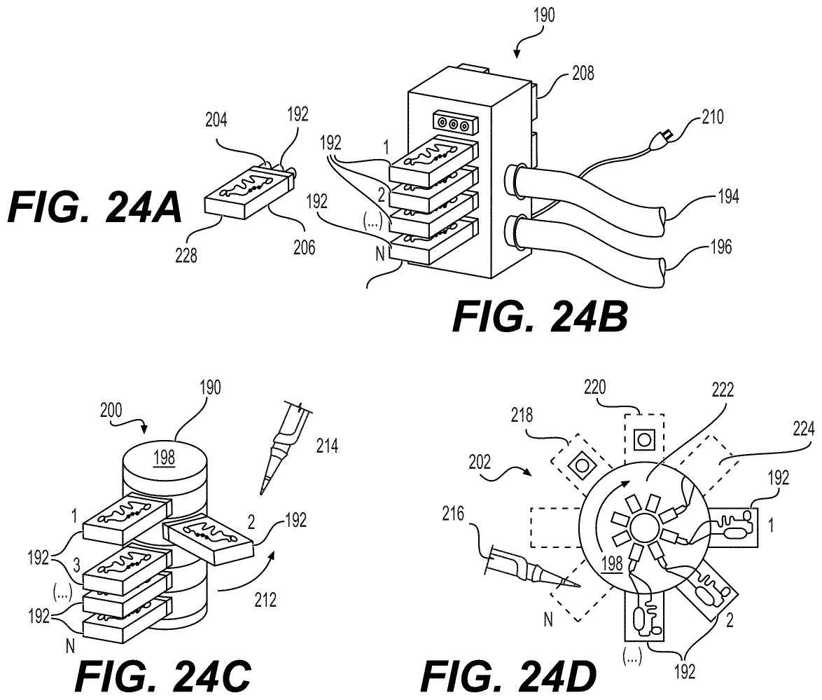

As shown in FIGS. 24A-24D, microfluidic chips (FIG. 24A) are inserted into electro pneumatic manifolds 190, 200 for stacking microfluidic devices 192 vertically 192 (FIG. 24B) or on a rotary mechanism 200 (FIG. 24C, FIG. 24D). A vertical manifold 190, 200 retains ideal gravitational orientation for each microfluidic device 192 and features quick connection to the pneumatics. A vertical tower 200 (FIG. 24C) can feature a rotating mechanism to allow for devices access while the microfluidic device 192 is still connected to the pneumatics. A carousel 202 (FIG. 24D) could also be implemented, where microfluidic devices 192 are connected radially around the control unit 198. Locations around the control unit allow for device manipulation and/or imaging.

A quick connector can be incorporated into the design so that chips can be added or removed easily. A rotating platform may also be integrated with imaging systems so that the chips can be autonomously imaged and analyzed.

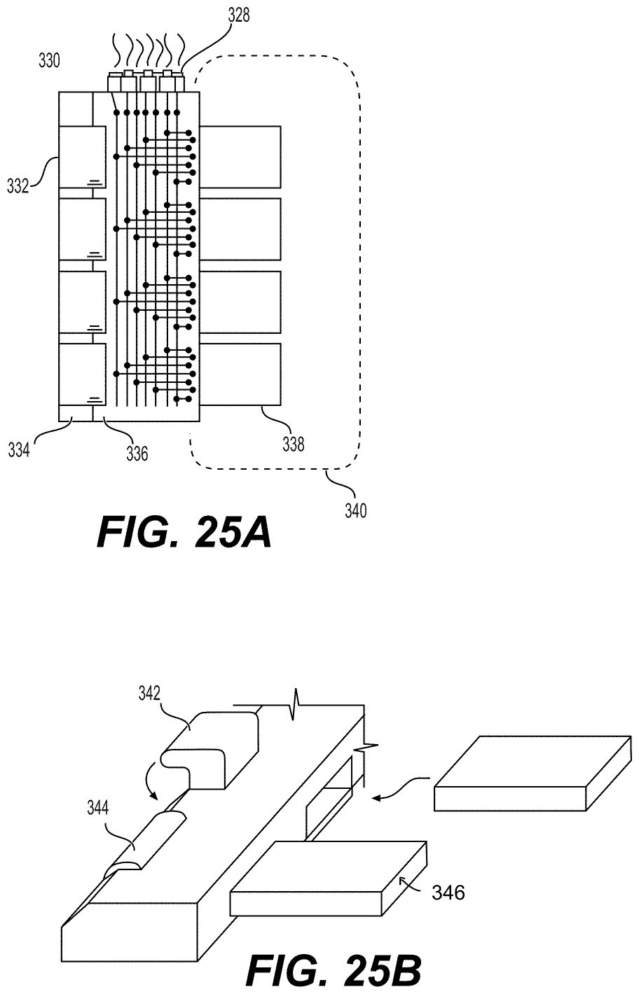

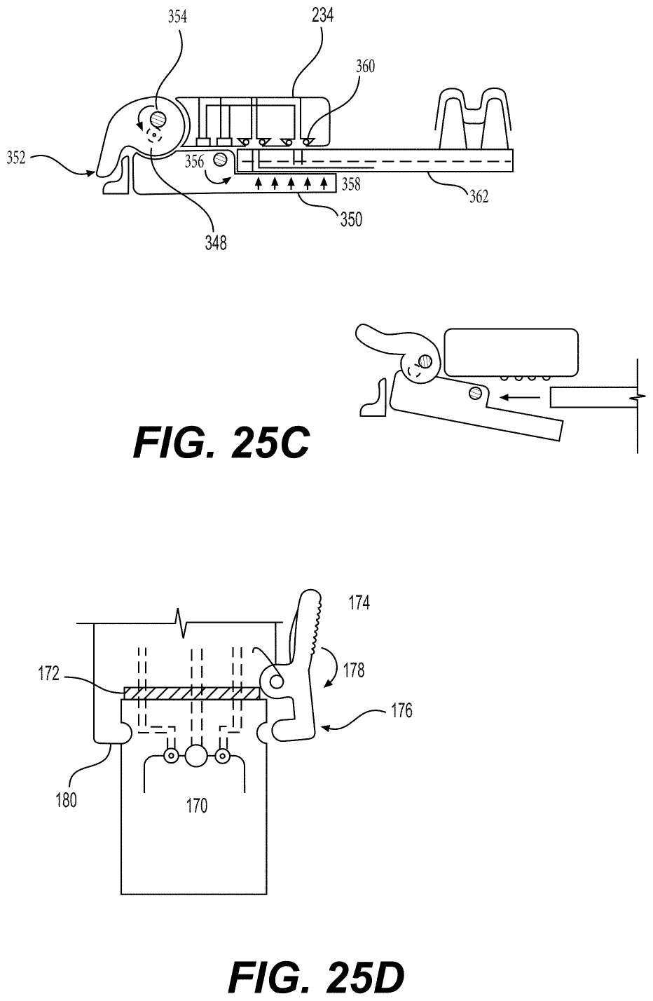

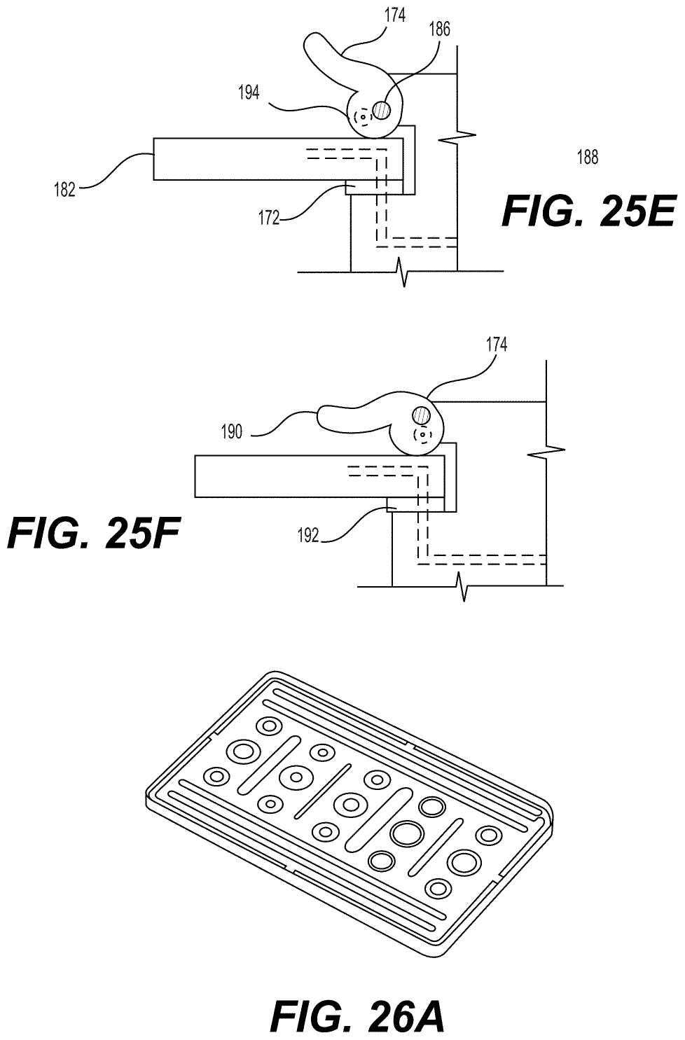

FIG. 25A-25F show exemplary quick connect devices for securing the microfluidic devices in the manifold. Lever (21) is pushed down to rotate axis (31) lobe. Off axis rotation of lobe causes rotation about (41). To provide clamp force (51) and compress O-ring (61) ensuring pneumatic connection to chip (71).

B. Microchip Devices with Features to Enhance Assembly

Microchip devices require channels for fluid flow, permeable membranes, connectors to channels for fluid intake and outflow, and configurations for culture of cells.

It is important that the membrane be bonded within the chip so that it does not leak, become detached during processing, and that the membrane bonds reliably and allow for gas to escape during the bonding process.

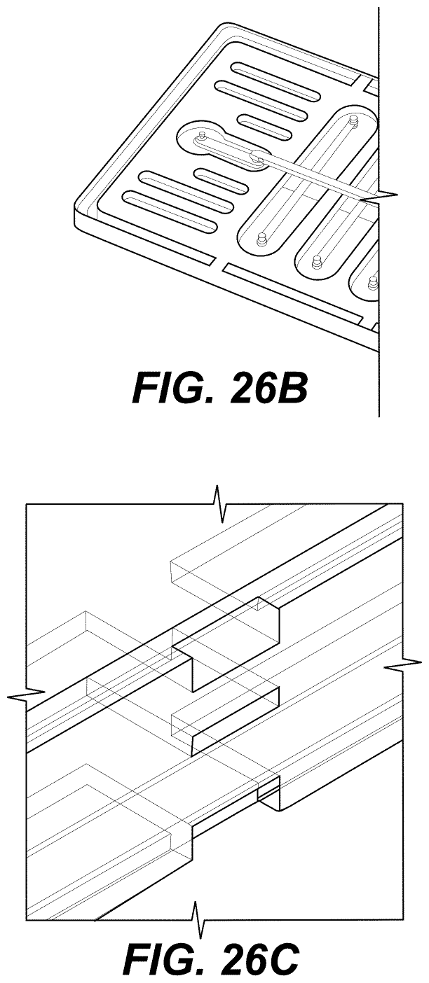

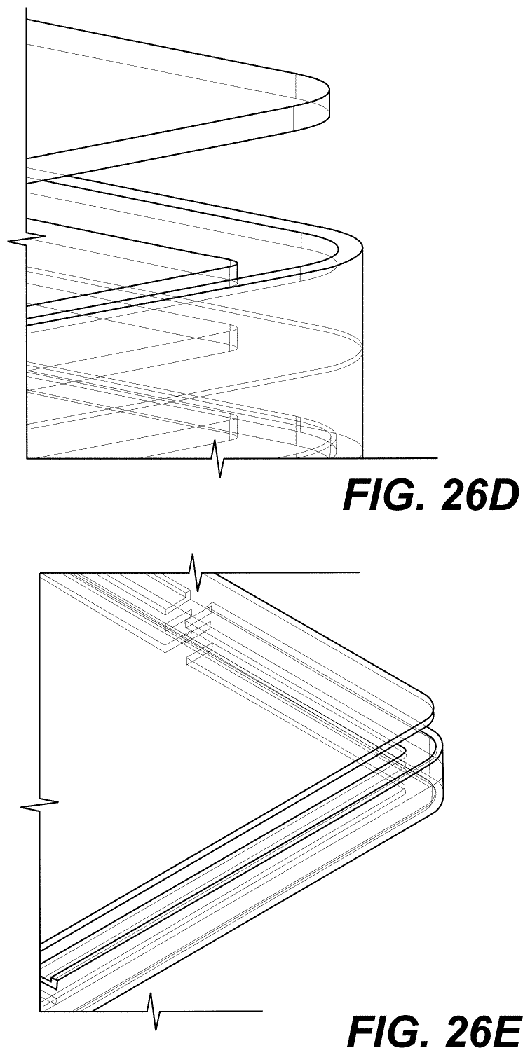

In a preferred embodiment, unlike the prior art devices which are patterned on a standard microscope slide (glass, 25.5×75.5 mm), these chips are 25 mm wide by 40 mm long (INSERT RANGE, RATIO AND ASPECT OF MEASUREMENTS), and are round on the corners (chamfered) (FIG. 26A). The shape facilitates alignment in the manifold, and makes the bonded membrane more resistant to being dislodged accidentally. The device as a thickness of 2-3 mm which it contains five layers. The size and shape of this chip are important because the reduced aspect ratio of length and width makes the chip less sensitive to flatness and runout of the bonding plane. Issues or parallelism between the two bonded halves of the chip are less relevant with the reduced aspect ratio.

FIG. 26A shows an example of a 25×40×2 mm chip with an integrated E-140 membrane in the middle.

FIG. 26B depicts a venting system in the chip of FIG. 26A to allow for gas escape during bonding. The chip format also includes small flat surfaces to improve the reliability of the bonding process and eliminate the presence of trapped bubbles and particles in the bonding process. The bonded chip is still strong and less likely to delaminate under heated conditions of applied stresses. In addition, the small bonding areas create open gas pockets in the center of the chip. The gas in these pockets can escape through the edges of the chip using small venting features. Without these vents the gasses inside can build up pressure and delaminate the chip.

FIG. 26C is in a CAD model showing the vents on a 5-layer chip. FIG. 26D-27E is a perspective view showing protective edges on the chip. The chip has a raised edge that protects the optical film on the top and bottom: Without these edges the film can lift up when it hits an object and delaminate the optical film. Se the corner of this chip with an unprotected edge.

We claim:

1. A microfluidic device comprising cyclic olefin copolymer membranes.

2. The device of claim 1 comprising a cyclic olefin copolymer membrane which is optically clear.

3. The device of claim 1 wherein the cyclic olefin copolymer is an elastomer.

4. The device of claim 1 wherein the device is a microfluidic chip for culturing or testing of cells or products thereof.

5. The device of claim 1 wherein the device is selected from the group consisting of pumps, valves, accumulators, pressure regulators, oxygenators, and pressure sensors

6. A method for bonding membranes made of cyclic olefin copolymers for use in microfluidic chips comprising
placing a cyclic olefin copolymer film onto a non-interactive carrier film, optionally formed of a polymer such as a biaxially oriented polyethylene terephthalate, supported by a flat substrate,
aligning a rigid component of a microfluidic chip with the carrier film and substrate, and
passing the rigid component with aligned film through a thermal laminator, or exposing to a thermal press or hot plate.

7. The method of claim 6 for bonding multiple membranes comprising using a roll extrusion process and cutting the bonded film to size using laser fabrication.

8. A water assisted laser machining method for etching elastomeric polymer film comprising using capillary action of a water film to secure the cut pieces in place.

9. The method of claim 8 further comprising providing a thermal sink and/or heat or infrared absorbing layer to control excess heat in the laser machining process.

10. A method for molding or shaping a thermoplastic elastomeric membrane comprising applying the membrane to a porous vacuum chuck with negative features, applying vacuum and heat, to mold the thermoformed elastomer membrane.

11. The method of claim 10 wherein the membrane is formed of cyclic olefin copolymer.

12. The method of claim 10 wherein the membrane is a component of the microfluidic device of claim 1.

13. A rolling elastomeric diaphragm for use in microfluidic valves and pump diaphragms, having high displacement from 0.2 to 3 millimeters with limited elastic deformation at a maximum of 10 percent strain.

14. The diaphragm of claim 13 shaped for use in a device component selected from the group consisting of external rolling diaphragms, internal rolling diaphragms, shape changing diaphragms, sideways rolling diaphragms, diaphragm micropumps, pressure sensors, and pressure accumulators.

15. The diaphragm of claim 14 in a pump comprising a pump chamber comprising a rolling diaphragm and a pump chamber with a deterministic displacement stroke that can displace a fixed volume with less that 5 percent error.

16. The diaphragm of claim 13 in a device where the diaphragm can be actuated using compressed gas and/or vacuum.

17. A microfluidic pressure regulator comprising a pneumatically actuated elastic membrane as a sealing feature and compressed gas as a biasing element.

18. The regulator of claim 17 structured to function as a back-pressure regulator.

19. The regulator of claim 18 wherein the regulator controls the fluid pressure downstream of the regulator, wherein the membrane has a low stiffness of 20-80 Mpa and an elongation at break greater than 500 percent so that it is not sensitive to strain energy in the membrane, wherein the fluid begins to flow once the fluid pressure exceeds the sealing pressure, optionally wherein the fluid pressure can be regulated by adjusting the compressed gas source and the flow can be stabilized by adding compliance in the fluidic circuit.

20. Microfluidic accumulators which store pressurized fluid in a microfluidic chip selected from the group consisting of accumulators using a flexible membrane to store pressure using stored elastic energy in the membrane, microfluidic accumulators using small dead-end microfluidic channels for trapping gas bubbles and storing volume under pressure, and microfluidic accumulators using a rolling diaphragm pressurized with air on one side and fluid stored in a reservoir.

21. Microfluidic pressure sensor comprising an optical level or change in capacitance and deformable membrane, where deformation of the elastic membrane occurs with an increase in pressure, optionally comprising optical means to measure the length of trapped gas bubbles in microfluidic channels which is proportional to the channel pressure.

22. A method of making hydrogels in a microfluidic device comprising providing movable, removable or dissolvable support structures are used to position the hydrogel at the time of formation, and/or to create channels in the hydrogel for fluid flow, optionally comprising polytetrafluoroethyelene (“PTFE”) allows for these structures to be removed without damaging the hydrogel after polymerization.

23. The method of claim 22 comprising dissolvable or removable structures to position or secure the hydrogel within the microfluidic device.

24. The method of claim 22 wherein the device comprises movable flaps to shape the hydrogel.

25. The method of claim 22 wherein the devices comprises structures for insertion and/or positioning in a manifold into which they are inserted.

26. The method of claim 22 wherein the hydrogel is held in place by surface tension and used to separate media channel and/or change flow configurations as a function of swelling.

27. A microfluidic device produced by the method of claim 22.

28. Removable caps for use in microfluidic devices for cell culture are selected from the group of caps comprising optically clear windows, elastomeric features for better compliance, and an adhesive pattern on a film for improved sealing.

29. A quick release top for a microfluidic chip comprising a gasket compressed using a spring-loaded lever, a toggle clamp or an overcenter latch.

30. Electro pneumatic manifolds comprising pneumatic lines, the manifolds stacking microfluidics devices vertically or on a rotary mechanism, comprising a latching system to enable quick connection of the microfluidic devices to the pneumatic lines.
